(19) United States
(12) Patent Application Publication
(10) Pub. No.: US 2014/0012115 A1
(43) Pub. Date: Jan. 9, 2014

(54) PLASMA DEPOSITED ADHESION PROMOTER LAYERS FOR USE WITH ANALYTE SENSORS

(71) Applicants: Yiwen Li, Arcadia, CA (US); Jenn-Hann Larry Wang, Northridge, CA (US); Rajiv Shah, Rancho Palos Verdes, CA (US)

(72) Inventors: Yiwen Li, Arcadia, CA (US); Jenn-Hann Larry Wang, Northridge, CA (US); Rajiv Shah, Rancho Palos Verdes, CA (US)

(73) Assignee: MEDTRONIC MINIMED, INC., Northridge, CA (US)

(21) Appl. No.: 13/541,262

(22) Filed: Jul. 3, 2012

(51) Int. Cl. A61B 5/1473; C23C 16/50

(52) U.S. Cl. 600/347; 427/2.12

(57) ABSTRACT

Embodiments of the invention provide methods and materials for making analyte sensors having a plurality of layered elements such as amperometric glucose sensors that are used by diabetic individuals to monitor blood sugar concentrations. Embodiments of the invention utilize plasma deposition technologies to form thin films of adhesion promoting compositions useful in such sensors. Sensors that incorporate the thin film compositions formed by these processes exhibit a number of desirable characteristics.

### BACKGROUND OF THE INVENTION

1. Field of the Invention

This invention relates to biosensors such as glucose sensors used in the management of diabetes and in particular methods and materials used to make such sensors.

2. Description of Related Art

Analyte sensors such as biosensors include devices that use biological elements to convert a chemical analyte in a matrix into a detectable signal. There are many types of biosensors used to detect wide variety of analytes. Perhaps the most studied type of biosensor is the amperometric glucose sensor, an apparatus commonly used to monitor glucose levels in individuals with diabetes.

A typical glucose sensor works according to the following chemical reactions:

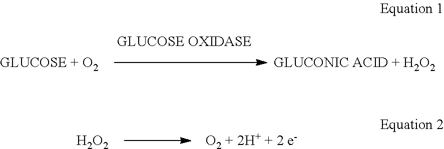

The glucose oxidase is used to catalyze the reaction between glucose and oxygen to yield gluconic acid and hydrogen peroxide as shown in equation 1. The H₂O₂ reacts electrochemically as shown in equation 2, and the current is measured by a potentiostat. The stoichiometry of the reaction provides challenges to developing in vivo sensors. In particular, for optimal sensor performance, sensor signal output should be determined only by the analyte of interest (glucose), and not by any co-substrates (O₂) or kinetically controlled parameters such as diffusion. If oxygen and glucose are present in equimolar concentrations, then the H₂O₂ is stoichiometrically related to the amount of glucose that reacts at the enzyme; and the associated current that generates the sensor signal is proportional to the amount of glucose that reacts with the enzyme. If, however, there is insufficient oxygen for all of the glucose to react with the enzyme, then the current will be proportional to the oxygen concentration, not the glucose concentration. Consequently, for the sensor to provide a signal that depends solely on the concentrations of glucose, glucose must be the limiting reagent, i.e. the O₂ concentration must be in excess for all potential glucose concentrations. A problem with using such glucose sensors in vivo, however, is that the oxygen concentration where the sensor is implanted in vivo is low relative to glucose, a phenomena which can compromise the accuracy of sensor readings.

Certain sensor designs address the oxygen deficit problem by using a series of layered materials selected to have specific function properties, for example an ability to selectively modulate the diffusion of analytes. Problems associated with such designs can include, for example, sensor layers delaminating and/or degrading over time in a manner that can limit the functional lifetime of the sensor. Methods and materials designed to address such challenges in this technology are desirable.

### SUMMARY OF THE INVENTION

Embodiments of the invention include dry plasma processes form making adhesion promoting (AP) layers in sensors comprising a plurality of layered materials. The dry plasma processes disclosed herein have a number of advantages over conventional wet chemistry processes used to form adhesion promoting layers, including reducing and/or eliminating the use of certain hazardous compounds, thereby reducing toxic wastes that can result from such processes. Embodiments of the invention also include adhesion promoting compositions formed from these processes, compositions that exhibit a combination of desirable material properties including relatively thin and highly uniform structural profiles. As disclosed below, amperometric glucose sensors that incorporate such adhesion promoting compositions exhibit a number of desirable characteristics.

Illustrative embodiments of the invention include methods of making an analyte sensor apparatus from a plurality of layered materials including an adhesion promoting layer formed from a dry plasma process. Typically these methods include the steps of providing a base layer, forming a conductive layer that includes at least one electrode over the base layer, forming an analyte sensing layer (e.g. one comprising glucose oxidase) over the conductive layer, and then forming an adhesion promoting layer over the analyte sensing layer using a plasma vapor deposition process. Optionally, the plasma vapor deposition process used to form the adhesion promoting layer is a pulse deposition process. In typical embodiments of the invention, the adhesion promoting layer comprises hexamethyldisiloxane (HMDSO). In some embodiments of the invention, the adhesion promoting layer comprises both hexamethyldisiloxane and allylamine, and is formed over the analyte sensing layer using a dual plasma vapor deposition process. In these embodiments, the hexamethyldisiloxane and the allylamine can be disposed in the adhesion promoting layer in a ratio that ranges from 5:1 to 1:1.

Embodiments of the invention include additional methodological steps for making analyte sensors from a plurality of layered materials, for example the step(s) of forming additional layers under and/or over the adhesion promoting layer discussed above, for example, analyte modulating layers, cover layers and the like. In one such embodiment, the method includes forming a protein layer over an analyte sensing layer, forming the adhesion promoting layer over this protein layer and then forming an analyte modulating layer over the adhesion promoting layer. Embodiments of the invention also include additional steps for varying the processes of the invention, for example, by performing one or pretreatment steps on one or more layer(s) over which the adhesion promoting layer is deposited. In an illustrative embodiment, one or more layer(s) over which the adhesion promoting layer is exposed to a gas plasma prior to depositing the hexamethyldisiloxane adhesion promoting composition. Embodiments of the invention can also include modifying the hexamethyldisiloxane composition after it is deposited, for example, by exposing the adhesion promoting layer to a crosslinking step or process after it is deposited. One illustrative crosslinking steps comprises exposing the hexamethyldisiloxane composition to a crosslinking gas plasma. Optionally in these steps, the gas plasma comprises a Helium plasma or an Oxygen plasma. Embodiments of the invention can also include performing a wash step on the analyte sensor following the crosslinking step and prior to forming the analyte modulating layer over the adhesion promoting layer.

Another embodiment of the invention is an analyte sensor apparatus comprising a plurality of layered materials including an adhesion promoting layer formed from a hexamethyldisiloxane composition using a plasma vapor deposition process. In certain embodiments of the invention, the adhesion promoting layer is formed to have a specific structure, for example, to an average thickness of less than 60, 50 or 40 nanometers. In some embodiments of the invention, the adhesion promoting composition is formed to have specific material properties and, for example, comprises both hexamethyldisiloxane and allylamine combined in a ratio of about 5:1, 4:1, 3:1, 2:1 or 1:1. In certain embodiments of the invention, one or more atoms in hexamethyldisiloxane and one or more atoms in allylamine are covalently crosslinked together. Illustrative sensors that include such adhesion promoting layers include amperometric glucose sensors that comprise glucose oxidase (e.g. within an analyte sensing layer) that is disposed over a working electrode (e.g. one disposed on a conductive layer). In some embodiments of the invention, the layers are organized so that an analyte sensing layer disposed over a conductive layer and the adhesion promoting layer disposed over the analyte sensing layer. Embodiments of the invention include additional layers and/or layers at different relative positions in the layered stack. Optionally, for example, the adhesion promoting layer is disposed over a protein layer that is disposed over the analyte sensing layer (e.g. a protein layer comprising bovine serum albumin or human serum albumin). Embodiments on the invention include layers disposed over the adhesion promoting layer, for example an analyte modulating layer that comprises a membrane designed to limit the diffusion of glucose. Certain embodiments of the invention include an analyte modulating layer that comprises an isocyanate compound, optionally one that comprises an atom that is covalently coupled to an atom in an allylamine compound that is disposed in the adhesion promoting layer.

Other embodiments of the invention include methods of sensing analytes within the body of a mammal using an amperometric sensor that comprises a plurality of layered materials including an adhesion promoting layer formed from plasma vapor deposited hexamethyldisiloxane (and optionally allylamine) as disclosed herein. Typically the methods comprise implanting the analyte sensor into the mammal, sensing an alteration in current at a sensor electrode in the presence of the analyte; and correlating the alteration in current with the presence and/or concentration of the analyte. In illustrative embodiments, the sensor is a glucose sensor used by a diabetic patient.

Other objects, features and advantages of the present invention will become apparent to those skilled in the art from the following detailed description. It is to be understood, however, that the detailed description and specific examples, while indicating some embodiments of the present invention are given by way of illustration and not limitation. Many changes and modifications within the scope of the present invention may be made without departing from the spirit thereof, and the invention includes all such modifications.

### BRIEF DESCRIPTION OF THE FIGURES

FIGS. 1A and 1B provide diagrams illustrating a sensor embodiment comprising a plurality of layered elements (in cross section).

FIG. 2 provides a diagram illustrating a sensor embodiment comprising a plurality of layered elements identified by numerals (in cross section).

FIG. 3 provides a diagram illustrating a schematic of a plasma AP process on a sensor plate.

FIG. 4 provides a perspective view illustrating a subcutaneous sensor insertion set, a telemetered characteristic monitor transmitter device, and a data receiving device embodying features of the invention.

FIG. 5 provides data from studies of sensors in an in vitro bicarbonate buffer testing system (BTS) that is designed to mimic in vivo conditions. In this system, sensor current is measured periodically in the presence of known concentrations of glucose and glucose values are then correlated with Isig, that is sensor current (in μA). This graph provides data (Isig over periods of time) from experiments using sensors constructed to include a plasma deposited AP layer comprising: HMDSO/allylamine (1:1 ratio, plasma process comprising equal vapor flow rate of two precursors). The results of this 3-day-long in vitro test show that these sensors exhibit a good starting Isig at 100 mg/dl glucose levels, very small sensor to sensor variations, as well as stable Isig (e.g. no drift upwards) even at the end of the test. The data from these tests provides evidence that sensors formed with these plasma deposited AP layers exhibit functional characteristics that are comparable, if not better than, sensors formed with conventional wet chemistry AP layers.

FIG. 6 provides data from studies of sensors in another in vitro sensor test system (SITS) that is designed to mimic in vivo conditions. This graph provides data (Isig over periods of time) using sensors constructed to include a plasma deposited AP layer comprising: HMDSO/allylamine (1:1 ratio, plasma process comprising equal vapor flow rate of two precursors). This 7-day-long standard sensor in vitro test result show that these sensors passed 4 calibration tests at different glucose levels, as well as oxygen response tests, temperature response tests, and Isig stability tests with limited sensor-to-sensor variation.

FIG. 7 provides a graph of BTS data (Isig over periods of time) using sensors constructed to include a plasma deposited AP layer comprising: HMDSO/allylamine (5:1 ratio, plasma process comprising rationed vapor flow rate of two precursors). The results of this 3-day-long in vitro test show that these sensors exhibit a good starting Isig at 100 mg/dl glucose levels, very small sensor to sensor variations, as well as stable Isig (e.g. no drift upwards) during the whole test. The data from these tests provides evidence that sensors formed with these plasma deposited AP layers exhibit functional characteristics that are comparable, if not better than, sensors formed with conventional wet chemistry AP layers.

FIG. 8 provides a graph of BTS data (Isig over periods of time) using sensors constructed to include a plasma deposited AP layer comprising: HMDSO (alone, without allylamine). Results of this 4-day-long in vitro test indicate that those sensors also had good starting Isig at 100 mg/dl glucose level, small sensor to sensor variations and stable Isig (no drift upward) during the whole test. The data from these tests provides evidence that sensors formed with these plasma deposited AP layers exhibit functional characteristics that are comparable, if not better than, sensors formed with conventional wet chemistry AP layers.

FIG. 9 provides a graph of in vivo data obtained from a non diabetic dog (sensor blood glucose mg/dL and sensor Isig-nA over 3 days of implantation using sensors constructed to include a plasma deposited AP layer comprising: HMDSO/allylamine (1:1 ratio, plasma process comprising equal vapor flow rate of two precursors). The graph shows that the sensor had fast run-in, stable Isig, low MARD (“mean absolute relative difference”, about 18%, an indication of low deviation), and a strong Isig even after 3 days of implantation.

FIG. 10 provides a graph of in vivo data obtained from a diabetic dog (sensor blood glucose mg/dL/sensor Isig-nA over 3 days of implantation using sensors constructed to include a plasma deposited AP layer comprising: HMDSO/allylamine (1:1 ratio, plasma process comprising equal vapor flow rate of two precursors). The graph shows that the sensor follows the in vivo glucose level changes very well in a diabetic dog from very beginning to the end of the implantation, significantly with great linearity (R=0.98) and low deviation (MARD=7%).

FIG. 11 shows data from a Fourier transform infrared spectroscopy (FTIR) study of plasma deposited AP. FIG. 11 shows 3 FTIR spectra respectively obtained from samples related to three different shelves in a plasma deposition chamber in a single run. There is no obvious different between those graphs, data which provides evidence for the uniformity of the plasma AP process.

FIGS. 12A and 12B provides graphs of data that illustrate desirable sensor performance profiles that can be obtained with sensors constructed to include a plasma deposited AP layer comprising HMDSO (alone, without allylamine). The top graph in FIG. 12A provides data showing stable sensor Isig over 2 days of implantation in human body with good in vivo sensor startup. The middle graph in FIG. 12A provides data showing Cal factor (Cal ratio) over the 2 day implantation. The Cal Factor started below 4 and stabilized around 4, confirming the desirable characteristics of these sensors. The bottom graph in FIG. 12A provides data showing sensor blood glucose follows actual body glucose change very well over the 2 day implantation in human body with good low MARD (11%). The top graph in FIG. 12B shows stable sensor Isig over 2 days of implantation in human body with good in vivo sensor startup. The middle graph in FIG. 12B shows good startup and stable Cal factor (less than 4), confirming a desirable Isig without any in vivo Isig dip issues. The bottom graph in FIG. 12B shows very good low MARD (about 11%), indicating very limited deviation between sensed glucose and actual blood glucose.

### DETAILED DESCRIPTION OF THE EMBODIMENTS

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art. All publications mentioned herein are incorporated herein by reference to disclose and describe the methods and/or materials in connection with which the publications are cited (see, e.g. Harsch et al., Journal of Neuroscience Methods 98 (2000) 135-144; Yoshinari et al., Biomedical Research 27(21): 29-36 (2006); U.S. Pat. No. 7,906,217 and United States Patent Application No. 20070202612). Publications cited herein are cited for their disclosure prior to the filing date of the present application. Nothing here is to be construed as an admission that the inventors are not entitled to antedate the publications by virtue of an earlier priority date or prior date of invention. Further the actual publication dates may be different from those shown and require independent verification.

It must be noted that as used herein and in the appended claims, the singular forms “a”, “and”, and “the” include plural referents unless the context clearly dictates otherwise. All numbers recited in the specification and associated claims that refer to values that can be numerically characterized with a value other than a whole number (e.g. “60 nanometers”) are understood to be modified by the term “about”.

The term “sensor,” as used herein, is a broad term and is used in its ordinary sense, including, without limitation, elements of an analyte-monitoring device that detect an analyte. In one embodiment, the sensor includes an electrochemical cell that has a working electrode, a reference electrode, and a counter electrode (e.g. a plurality of each of these electrodes) passing through and secured within the sensor body forming an electrochemically reactive surface at one location on the body, an electronic connection at another location on the body, and a membrane system affixed to the body and covering the electrochemically reactive surface. During general operation of the sensor, a biological sample (for example, blood or interstitial fluid), or a portion thereof, contacts (directly or after passage through one or more membranes or domains) an enzyme (for example, glucose oxidase); the reaction of the biological sample (or portion thereof) results in the formation of reaction products that allow a determination of the analyte level in the biological sample.

Embodiments of the invention disclosed herein provide sensors of the type used, for example, in subcutaneous or transcutaneous monitoring of blood glucose levels in a diabetic patient. A variety of implantable, electrochemical biosensors have been developed for the treatment of diabetes and other life-threatening diseases. Many existing sensor designs use some form of immobilized enzyme to achieve their bio-specificity. Embodiments of the invention described herein can be adapted and implemented with a wide variety of known electrochemical sensors, including for example, U.S. Patent Application No. 20050115832, U.S. Pat. Nos. 6,001,067, 6,702,857, 6,212,416, 6,119,028, 6,400,974, 6,595,919, 6,141,573, 6,122,536, 6,512,939 5,605,152, 4,431,004, 4,703,756, 6,514,718, 5,985,129, 5,390,691, 5,391, 250, 5,482,473, 5,299,571, 5,568,806, 5,494,562, 6,120,676, 6,542,765 as well as PCT International Publication Numbers WO 01/58348, WO 04/021877, WO 03/034902, WO 03/035117, WO 03/035891, WO 03/023388, WO 03/022128, WO 03/022352, WO 03/023708, WO 03/036255, WO03/036310 WO 08/042,625, and WO 03/074107, and European Patent Application EP 1153571, the contents of each of which are incorporated herein by reference.

A wide variety of sensors and sensor elements are known in the art including amperometric sensors used to detect and/or measure biological analytes such as glucose. Many glucose sensors are based on an oxygen (Clark-type) amperometric transducer (see, e.g. Yang et al., Electroanalysis 1997, 9, No. 16: 1252-1256; Clark et al., Ann. N.Y. Acad. Sci. 1962, 102, 29; Updike et al., Nature 1967, 214,986; and Wilkins et al., Med. Engin. Physics, 1996, 18, 273.3-51). Electrochemical glucose sensors that utilize the chemical reaction between glucose and glucose oxidase to generate a measurable signal typically include polymeric compositions that modulate the diffusion of analytes including glucose in order to overcome what is known in the art as the “oxygen deficit problem”. Specifically, because glucose oxidase based sensors require both oxygen (O₂) as well as glucose to generate a signal, the presence of an excess of oxygen relative to glucose, is necessary for the operation of a glucose oxidase based glucose sensor. However, because the concentration of oxygen in subcutaneous tissue is much less than that of glucose, oxygen can be the limiting reactant in the reaction between glucose, oxygen, and glucose oxidase in a sensor, a situation which compromises the sensor's ability to produce a signal that is strictly dependent on the concentration of glucose. The modification and/or substitution of layered materials in a sensor can be problematical in that such modifications can result in unpredictable alterations in the crucial permselective properties of these layers. For example, because the properties of a material can influence the rate at which compounds diffuse through that material to the site of a measurable chemical reaction, the material properties of an analyte modulating layer used in electrochemical glucose sensors that utilize the chemical reaction between glucose and glucose oxidase to generate a measurable signal, should not for example, favor the diffusion of glucose over oxygen in a manner that contributes to the oxygen deficit problem. In this context, the plasma deposited hexamethyldisiloxane (and optionally allylamine) AP layers that are disclosed herein exhibit functional features including diffusion profiles that make them useful in layered sensor structures designed to address the oxygen deficit problem that is observed in amperometric glucose sensors. These materials can be used to make sensors having a number of desirable properties, including an extended shelf life as well as enhanced performance profiles.

As discussed in detail below, embodiments of the invention disclosed herein provide sensor elements having enhanced material properties and sensor systems (e.g. those comprising a sensor and associated electronic components such as a monitor, a processor and the like) constructed to include such elements. The disclosure further provides methods for making and using such sensors. While some embodiments of the invention pertain to glucose sensors, a variety of the processes and materials disclosed herein (e.g. adhesion promoting layers formed from plasma deposition processes) can be adapted for use with any one of the wide variety of analyte sensors known in the art. Such sensors of the invention exhibit a surprising degree of flexibility and versatility, characteristics which allow a wide variety of sensor configurations to be designed to examine a wide variety of analytes. The term “analyte” as used herein is a broad term and is used in its ordinary sense, including, without limitation, to refer to a substance or chemical constituent in a fluid such as a biological fluid (for example, blood, interstitial fluid, cerebral spinal fluid, lymph fluid or urine) that can be analyzed. Analytes can include naturally occurring substances, artificial substances, metabolites, and/or reaction products. In illustrative embodiments, the analyte is glucose.

Specific aspects of embodiments of the invention are discussed in detail in the following sections.

### Typical Processes, Elements, and Analyte Sensors of the Invention

As discussed in detail below, embodiments of the invention relate to methods for making and using an electrochemical sensor that exhibits a novel constellation of elements including an adhesion promoting layer having a unique set of features including improved material properties as well as an ease in manufacture. The electrochemical sensors of the invention are designed to measure a concentration of an analyte of interest (e.g. glucose) or a substance indicative of the concentration or presence of the analyte in fluid. In some embodiments, the sensor is a continuous device, for example a subcutaneous, transdermal, or intravascular device. In some embodiments, the device can analyze a plurality of intermittent blood samples to provide an output signal indicative of the concentration of the analyte of interest. Such sensors comprise one or more adhesion promoting layers having a constellation of selected material properties, including allowing glucose and oxygen to appropriately migrate through these layers prior to reacting with a sensing complex (e.g. an enzyme such as glucose oxidase disposed on an electrode). The presence of the analyte can be measured using electrochemical methods and the output of an electrode system can function as a measure of the analyte. Typically, the sensor is of the type that senses a product or reactant of an enzymatic reaction between an analyte and an enzyme in the presence of oxygen as a measure of the analyte in vivo or in vitro (e.g. hydrogen peroxide generated by glucose oxidase I he presence of glucose).

As noted above, embodiments of the invention relate to layered sensor structures, for example those found in amperometric glucose sensors having a plurality of layered materials including one or more adhesion promoting materials/layers. A major function of such AP layers is to inhibit layer delamination that can translate into poor sensor performance. However, these layers should be as thin as possible in amperometric sensors because the additional material bulk can alter sensor current profiles, a phenomena which can negatively impact sensor performance. While adhesion promoting materials/layers are known in the art, in many conventional sensor fabrication processes, an adhesion promoter layer is formed using hazardous chemicals such as glutaraldehyde. For example, some wet chemistry AP processes use glutaraldehyde to cross link compositions such as silanes (e.g. 3-aminopropyltriethoxysilanes) that are located between surrounding layers, for example those that comprise glucose oxidase (GOx) and/or serum albumin, and/or those that comprise polymers such as those found in glucose limiting membranes (“GLMs”). Unfortunately, there are a number of problems associated with the use of glutaraldehyde. One problem is that this compound can make AP layers unstable in the air. Another problem is that sensor signals tend to decreased over time due to ongoing crosslinking caused by existence of residual cross linker that can be present in the sensor membrane matrix. In addition, the handling of glutaraldehyde waste in an environmentally prudent manner can be quite costly. Consequently, reducing and/or eliminating glutaraldehyde usage in various steps that involve sensor fabrication has a number of benefits.

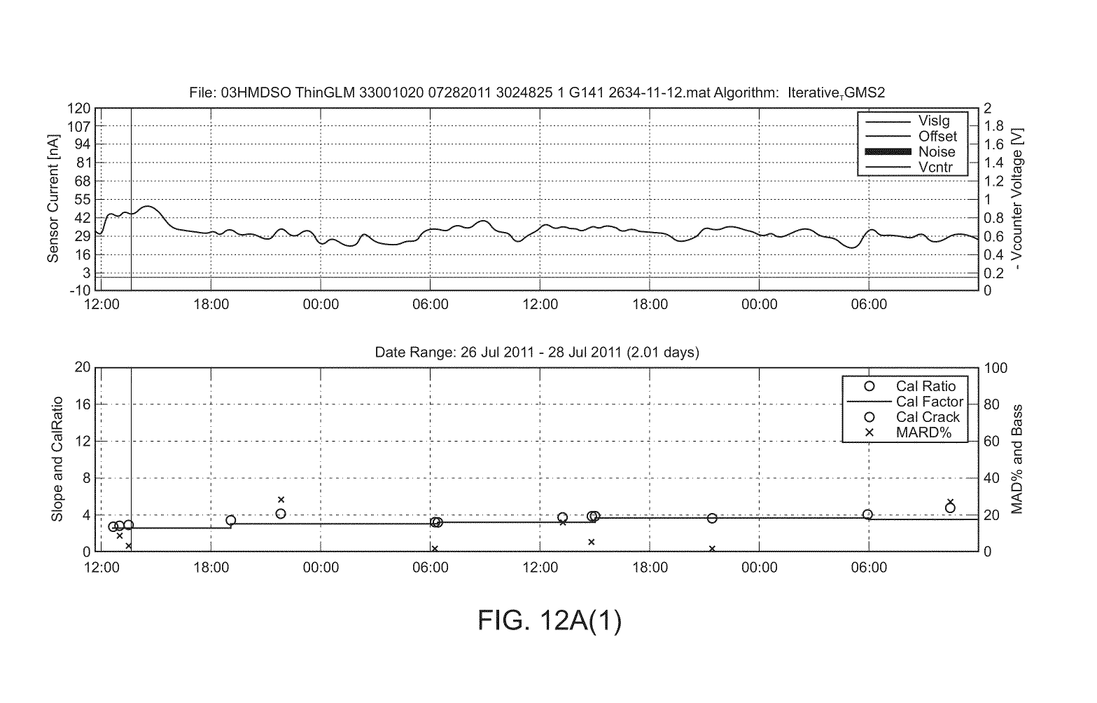

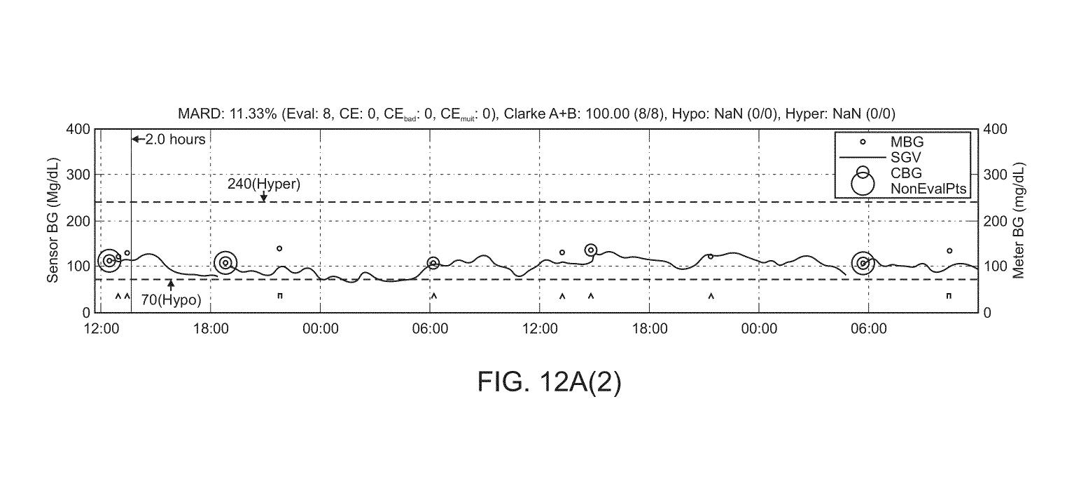

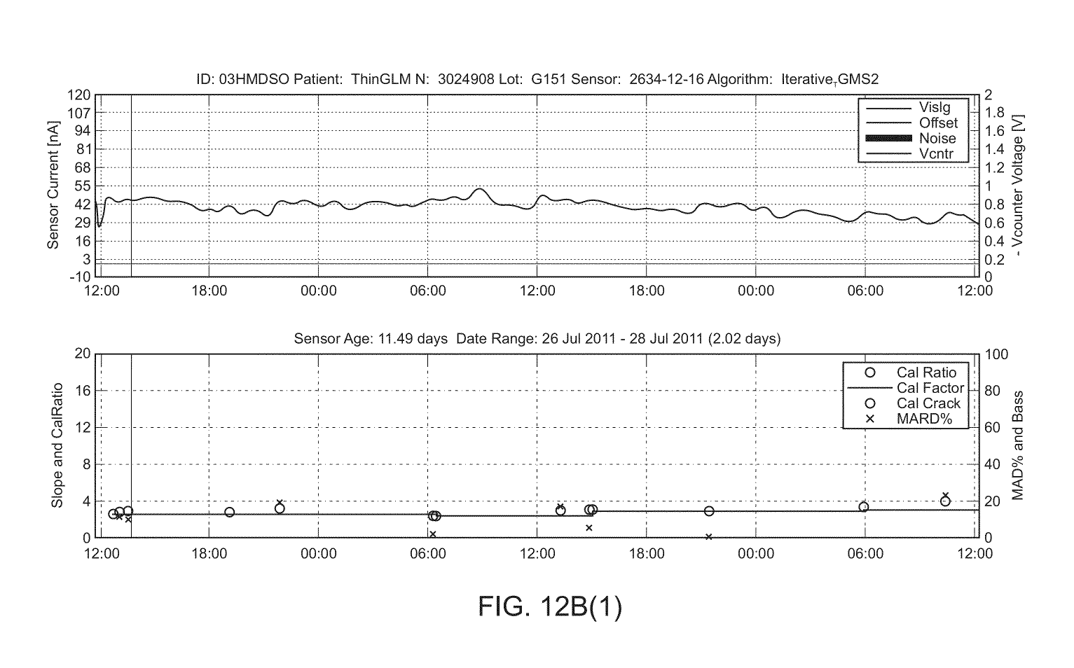

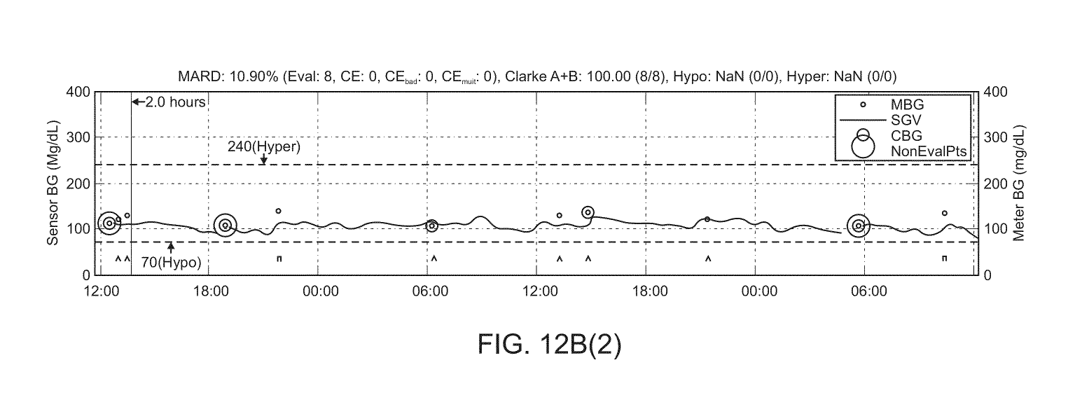

Embodiments of the invention include dry plasma processes used to form adhesion promoting layers in sensors comprising a plurality of layered materials. Such dry plasma processes have a number of advantages over conventional wet chemistry processes, including reducing or eliminating the need for certain biohazardous compounds (e.g. glutaraldehyde) that are used conventional processes, thereby reducing chemical wastes that result from production processes. Embodiments of the invention include adhesion promoting layer compositions formed from these processes, compositions that exhibit a combination of desirable material properties including a very thin, yet uniform structural profiles. Such adhesion promoting layers are particularly useful in the construction of electrochemical sensors for in vivo use. As disclosed below, amperometric glucose sensors that incorporate such adhesion promoting compositions exhibit a number of desirable characteristics including enhanced performance profiles (see, e.g. FIGS. 12A and 12B). Embodiments of the invention allow for a combination of desirable properties including: an enhanced lifetime profile as well as a permeability profile to molecules such as glucose that allow them to, for example address the oxygen deficit problem. In addition, these adhesion promoting layers can be formed using environmentally friendly and cost effective manufacturing processes.

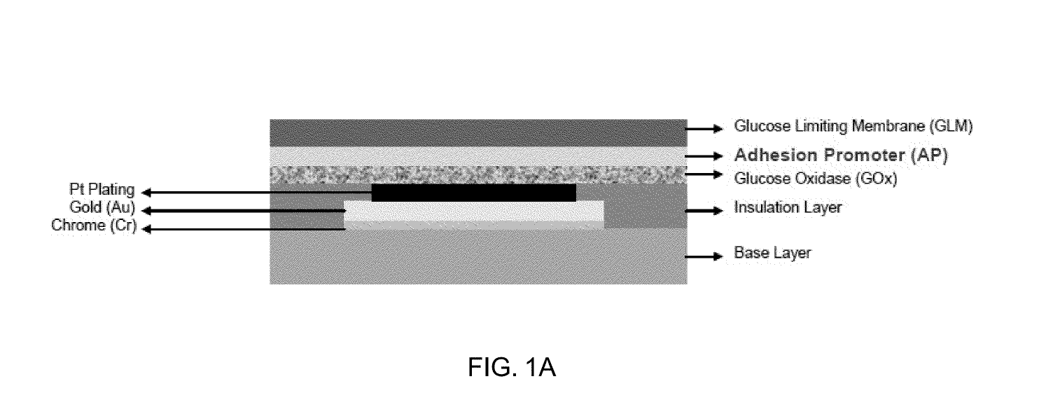

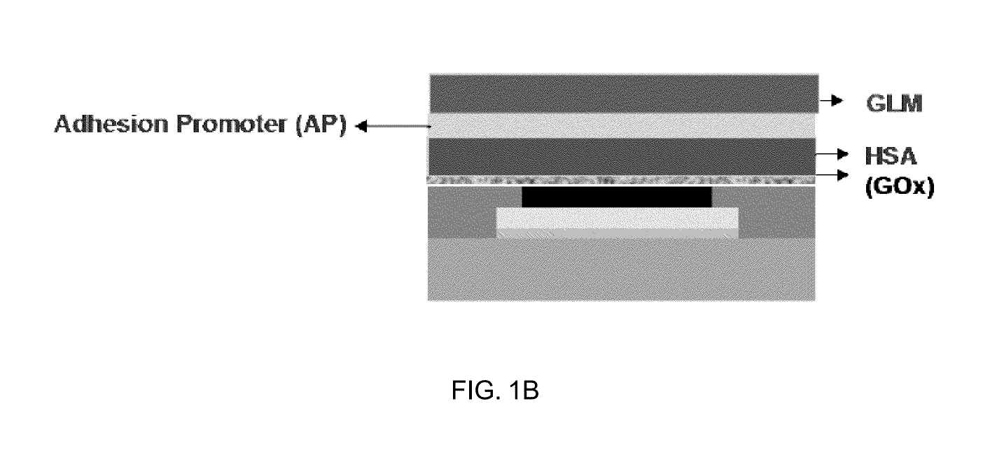

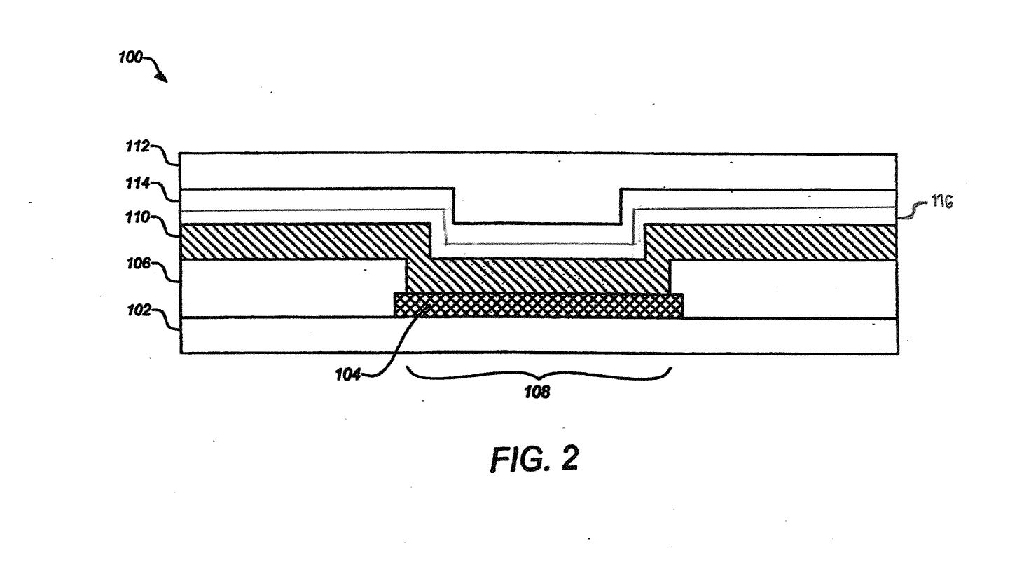

Illustrative embodiments of the invention include methods of making glucose sensor apparatus from a plurality of layered materials including an adhesion promoting layer formed from a dry plasma process (see, e.g. FIGS. 1 and 2). Typically these methods include the steps of providing a base layer, forming a conductive layer over the base that includes a working, counter and reference electrode, forming an analyte sensing layer (e.g. one comprising glucose oxidase) over the conductive layer, and then forming an adhesion promoting layer over the analyte sensing layer using a plasma vapor deposition process. Optionally the plasma vapor deposition process used to form the adhesion promoting layer is a pulse deposition process. In certain embodiments of the invention, the adhesion promoting layer comprises hexamethyldisiloxane. In some embodiments of the invention, the adhesion promoting layer comprises hexamethyldisiloxane and allylamine, and is formed over the analyte sensing layer using a dual plasma vapor deposition process. In these embodiments, the hexamethyldisiloxane and the allylamine are disposed in the adhesion promoting layer in a ratio of not more (or not less) than 5:1, 4:1, 3:1, 2:1 and 1:1.

Embodiments of the invention include additional methodological steps for making analyte sensors from a plurality of layered materials, for example the step(s) of forming additional layers under or over the adhesion promoting layer discussed above, for example, protein layers, analyte modulating layers, cover layers and the like. In one such embodiment, the method includes forming a protein layer over an analyte sensing layer, and then forming the adhesion promoting layer over this protein layer. Embodiments of the invention can also include performing a pretreatment step on one or more layer(s) over which the adhesion promoting layer is deposited, for example, by exposing the layer(s) to a pretreating gas plasma. Embodiments of the invention can also include performing a crosslinking step on the adhesion promoting layer after it is deposited, wherein the crosslinking step comprises exposure to a crosslinking gas plasma. Optionally in these steps, the pretreating or crosslinking gas plasma comprises a Helium plasma or an Oxygen plasma. In addition, embodiments of the invention can also include performing a wash step on the analyte sensor following the crosslinking step and prior to forming the analyte modulating layer over the adhesion promoting layer.

Another embodiment of the invention is an analyte sensor apparatus comprising a plurality of layered materials including an adhesion promoting layer having a constellation of material properties that result from it being formed from a hexamethyldisiloxane composition using a plasma vapor deposition process. Typically, this sensor is coupled to a structure adapted to be implanted in vivo (e.g. a needle, a catheter, a probe or the like). In some embodiments of the invention, the adhesion promoting layer comprises both hexamethyldisiloxane and allylamine combined in a ratio from 5:1 to 1:1 (e.g. 5:1, 4:1, 3:1, 2:1 or 1:1). In certain embodiments of the invention, the hexamethyldisiloxane and allylamine are covalently crosslinked together. In some embodiments, the adhesion promoting layer is formed to have a specific structure, for example, to an average thickness of less than 60, 50 or 40 nanometers, and/or to have relatively few (e.g. as compared to conventional AP layers formed from wet chemistry processes) or no holes or breaches in that span the layer (e.g. pinhole like structures in a portion of the adhesion promoting layer that expose a portion of an underlying layer). Typical sensors that include such adhesion promoting layers include amperometric glucose sensors that comprise glucose oxidase (e.g. within an analyte sensing layer) disposed over a working electrode. Specific illustrative examples of such embodiments are diagrammed in FIGS. 1A and 1B. In some embodiments of the invention, the layers are organized so that an analyte sensing layer is disposed over a conductive layer and the adhesion promoting layer disposed over the analyte sensing layer. In certain embodiments, the adhesion promoting layer is in direct contact with materials in the protein layer on a first side and in direct contact with materials in the analyte modulating layer on a second side. In other embodiments, the adhesion promoting layer is in direct contact with materials in the analyte sensing layer on a first side and in direct contact with materials in the analyte modulating layer on a second side. In some embodiments of the invention, the analyte sensing layer comprises an enzyme selected from the group consisting of glucose oxidase, glucose dehydrogenase, lactate oxidase, hexokinase and lactose dehydrogenase.

In some embodiments, the adhesion promoting layer is disposed over a protein layer that is disposed over the analyte sensing layer, for example a protein layer comprising bovine serum albumin (BSA) or human serum albumin (HSA). In typical embodiments, the protein constituent in this layer comprises an albumin such as human serum albumin. The HSA concentration may vary between about 0.5%-30% (w/v). Typically the HSA concentration is about 1-10% w/v, and most typically is about 5% w/v. In alternative embodiments of the invention, collagen or BSA or other structural proteins used in these contexts can be used instead of or in addition to HSA. Embodiments on the invention include further layers disposed over the adhesion promoting layer, for example an analyte modulating layer. In certain embodiments of the invention, an analyte modulating layer comprises an isocyanate compound and the isocyanate comprises an atom is covalently coupled to an atom in an allylamine in the adhesion promoting layer. In certain embodiments of the invention, the analyte modulating layer comprises a linear polyurethane/polyurea polymer. Typically, the analyte modulating layer is formed from a mixture comprising: a diisocyanate compound (typically about 50 mol % of the reactants in the mixture); at least one hydrophilic diol or hydrophilic diamine compound (typically about 17 to 45 mol % of the reactants in the mixture); and a siloxane compound. Optionally the polyurethane/polyurea polymer comprises 45-55 mol % (e.g. 50 mol %) of a diisocyanate (e.g. 4,4′-diisocyanate), 10-20 (e.g. 12.5 mol %) mol % of a siloxane (e.g. polymethylhydrosiloxane, trimethylsilyl terminated), and 30-45 mol % (e.g. 37.5 mol %) of a hydrophilic diol or hydrophilic diamine compound (e.g. polypropylene glycol diamine having an average molecular weight of 600 Daltons, Jeffamine 600). In certain embodiments of the analyte modulating layer a first polyurethane/polyurea polymer is blended with a second polymer formed from a mixture comprising: 5-45 weight % of a 2-(dimethylamino)ethyl methacrylate compound; 15-55 weight % of a methyl methacrylate compound; 15-55 weight % of a polydimethyl siloxane monomethacryloxypropyl compound; 5-35 weight % of a poly(ethylene oxide) methyl ether methacrylate compound; and 1-20 weight % 2-hydroxyethyl methacrylate, with the first polymer and the second polymer blended together at a ratio between 1:1 and 1:20 weight %.

In illustrative embodiments of the invention, the analyte modulating layer comprises a blended mixture of a polyurethane/polyurea polymer formed from a mixture comprising a diisocyanate; a hydrophilic polymer comprising a hydrophilic diol or hydrophilic diamine; and a siloxane having an amino, hydroxyl or carboxylic acid functional group at a terminus. Optionally this polyurethane/polyurea polymer is blended with a branched acrylate polymer formed from a mixture comprising a butyl, propyl, ethyl or methyl-acrylate; an amino-acrylate; a siloxane-acrylate; and a poly(ethylene oxide)-acrylate. Optionally the analyte modulating layer exhibits a water adsorption profile of 40-60% of membrane weight. In certain embodiments of the invention, the analyte modulating layer is 5-15 um thick. In some embodiments, the analyte modulating layer comprises a polyurethane/polyurea polymer formed from a mixture comprising: a diisocyanate; a hydrophilic polymer comprising a hydrophilic diol or hydrophilic diamine; a siloxane having an amino, hydroxyl or carboxylic acid functional group at a terminus; and a polyurethane/polyurea polymer stabilizing compound selected for its ability to inhibit thermal and oxidative degradation of polyurethane/polyurea polymers formed from the mixture, wherein the polyurethane/polyurea polymer stabilizing compound has a molecular weight of less than 1000 g/mol; and comprises a benzyl ring having a hydroxyl moiety (ArOH). In typical embodiments of the invention, the polyurethane/polyurea polymer stabilizing compound exhibits an antioxidant activity (e.g. embodiments that comprise phenolic antioxidants). Optionally, the polyurethane/polyurea polymer stabilizing compound comprises at least two benzyl rings having a hydroxyl moiety.

Other embodiments of the invention include methods of sensing analytes within the body of a mammal using an amperometric sensor that comprises a plurality of layered materials including an adhesion promoting layer formed from plasma vapor deposited hexamethyldisiloxane (and optionally allylamine) as disclosed herein. Typically the methods comprise implanting the analyte sensor into the mammal, sensing an alteration in current at a sensor electrode in the presence of the analyte; and correlating the alteration in current with the presence and/or concentration of the analyte. In illustrative embodiments, the sensor is a glucose sensor used by a diabetic patient (see, e.g. FIG. 12).

FIG. 2 illustrates a cross-section of one sensor embodiment 100 of the present invention. This sensor embodiment is formed from a plurality of components that are in the form of layers of various conductive and non-conductive constituents disposed on each other according to art accepted methods and/or the specific methods of the invention disclosed herein. The components of the sensor are typically characterized herein as layers because, for example, it allows for a facile characterization of the sensor structure shown in FIG. 2. Artisans will understand however, that in certain embodiments of the invention, the sensor constituents are combined such that multiple constituents form one or more heterogeneous layers. In this context, those of skill in the art understand that the ordering of the layered constituents can be altered in various embodiments of the invention.

The embodiment shown in FIG. 2 includes a base layer 102 to support the sensor 100. The base layer 102 can be made of a material such as a metal and/or a ceramic and/or a polymeric substrate, which may be self-supporting or further supported by another material as is known in the art. Embodiments of the invention include a conductive layer 104 which is disposed on and/or combined with the base layer 102. Typically the conductive layer 104 comprises one or more electrodes. An operating sensor 100 typically includes a plurality of electrodes such as a working electrode, a counter electrode and a reference electrode. Other embodiments may also include a plurality of working, counter and reference electrodes sets that are grouped together as units.

As discussed in detail below, the base layer 102 and/or conductive layer 104 can be generated using many known techniques and materials. In certain embodiments of the invention, the electrical circuit of the sensor is defined by etching the disposed conductive layer 104 into a desired pattern of conductive paths. A typical electrical circuit for the sensor 100 comprises two or more adjacent conductive paths with regions at a proximal end to form contact pads and regions at a distal end to form sensor electrodes. An electrically insulating cover layer 106 such as a polymer coating can be disposed on portions of the sensor 100. Acceptable polymer coatings for use as the insulating protective cover layer 106 can include, but are not limited to, non-toxic biocompatible polymers such as silicone compounds, polyimides, biocompatible solder masks, epoxy acrylate copolymers, or the like. In the sensors of the present invention, one or more exposed regions or apertures 108 can be made through the cover layer 106 to open the conductive layer 104 to the external environment and to, for example, allow an analyte such as glucose to permeate the layers of the sensor and be sensed by the sensing elements. Apertures 108 can be formed by a number of techniques, including laser ablation, tape masking, chemical milling or etching or photolithographic development or the like. In certain embodiments of the invention, during manufacture, a secondary photoresist can also be applied to the protective layer 106 to define the regions of the protective layer to be removed to form the aperture(s) 108. The exposed electrodes and/or contact pads can also undergo secondary processing (e.g. through the apertures 108), such as additional plating processing, to prepare the surfaces and/or strengthen the conductive regions.

In the sensor configuration shown in FIG. 2, an analyte sensing layer 110 is disposed on one or more of the exposed electrodes of the conductive layer 104. Typically, the analyte sensing layer 110 comprises an enzyme capable of producing and/or utilizing oxygen and/or hydrogen peroxide, for example the enzyme glucose oxidase. Optionally the enzyme in the analyte sensing layer is combined with a second carrier protein such as human serum albumin, bovine serum albumin or the like. In an illustrative embodiment, an oxidoreductase enzyme such as glucose oxidase in the analyte sensing layer 110 reacts with glucose to produce hydrogen peroxide, a compound which then modulates a current at an electrode. As this modulation of current depends on the concentration of hydrogen peroxide, and the concentration of hydrogen peroxide correlates to the concentration of glucose, the concentration of glucose can be determined by monitoring this modulation in the current. Such modulations in the current caused by changing hydrogen peroxide concentrations can by monitored by any one of a variety of sensor detector apparatuses such as a universal sensor amperometric biosensor detector or one of the other variety of similar devices known in the art such as glucose monitoring devices produced by Medtronic MiniMed. Typical sensor embodiments of this element of the invention utilize an enzyme (e.g. glucose oxidase) that has been combined with a second protein (e.g. albumin) in a fixed ratio (e.g. one that is typically optimized for glucose oxidase stabilizing properties) and then applied on the surface of an electrode to form a thin enzyme constituent. In a typical embodiment, the analyte sensing constituent comprises a GOx and HSA mixture. The enzyme and the second protein (e.g. an albumin) are typically treated to form a crosslinked matrix (e.g. by adding a cross-linking agent to the protein mixture). As is known in the art, crosslinking conditions may be manipulated to modulate factors such as the retained biological activity of the enzyme, its mechanical and/or operational stability. Illustrative crosslinking procedures are described in U.S. patent application Ser. No. 10/335,506 and PCT publication WO 03/035891 which are incorporated herein by reference. For example, an amine cross-linking reagent, such as, but not limited to, glutaraldehyde, can be added to the protein mixture.

In embodiments of the invention, the analyte sensing layer 110 can be applied over portions of the conductive layer or over the entire region of the conductive layer. Typically the analyte sensing layer 110 is disposed on the working electrode which can be the anode or the cathode. Optionally, the analyte sensing layer 110 is also disposed on a counter and/or reference electrode. While the analyte sensing layer 110 can be up to about 1000 microns (μm) in thickness, typically the analyte sensing layer is relatively thin as compared to those found in sensors previously described in the art, and is for example, typically less than 1, 0.5, 0.25 or 0.1 microns in thickness. As discussed in detail below, some methods for generating a thin analyte sensing layer 110 include brushing the layer onto a substrate (e.g. the reactive surface of a platinum black electrode), as well as spin coating processes, dip and dry processes, low shear spraying processes, ink-jet printing processes, silk screen processes and the like.

Typically, the analyte sensing layer 110 is coated and or disposed next to one or more additional layers. Optionally, the one or more additional layers includes a protein layer 116 disposed upon the analyte sensing layer 110. Typically, the protein layer 116 comprises a protein such as human serum albumin, bovine serum albumin or the like. Typically, the protein layer 116 comprises human serum albumin. In some embodiments of the invention, an additional layer includes an analyte modulating layer 112 that is disposed above the analyte sensing layer 110 to regulate analyte access with the analyte sensing layer 110. For example, the analyte modulating membrane layer 112 can comprise a glucose limiting membrane, which regulates the amount of glucose that contacts an enzyme such as glucose oxidase that is present in the analyte sensing layer. Such glucose limiting membranes can be made from a wide variety of materials known to be suitable for such purposes, e.g., silicone compounds such as polydimethyl siloxanes, polyurethanes, polyurea cellulose acetates, NAFION, polyester sulfonic acids (e.g. Kodak AQ), hydrogels, and the polyurea polymers and polymer blends disclosed herein.

In embodiments of the invention, an adhesion promoter layer 114 is disposed between layers such as the analyte modulating layer 112 and the analyte sensing layer 110 as shown in FIG. 2 in order to facilitate their contact and/or adhesion. In a specific embodiment of the invention, an adhesion promoter layer 114 is disposed between the analyte modulating layer 112 and the protein layer 116 as shown in FIG. 2 in order to facilitate their contact and/or adhesion. The adhesion promoter layer 114 can be made from any one of a wide variety of materials known in the art to facilitate the bonding between such layers. Typically, the adhesion promoter layer 114 comprises hexamethyldisiloxane or hexamethyldisiloxane and allylamine combined in a ratio from 5:1 to 1:1. Embodiments of typical elements that can be included in and/or adapted for use with the sensors disclosed herein are disclosed in U.S. Patent Application Publication Nos. 20070163894, 20070227907, 20100025238, 20110319734 and 20110152654, the contents of each of which are incorporated herein by reference. For example, FIG. 2 in U.S. Patent Application Publication No. 20070163894 provides a perspective view illustrating a subcutaneous sensor insertion set, a telemetered characteristic monitor transmitter device, and a data receiving device of the type that can be adapted for use with embodiments of the invention. In addition, number of articles, U.S. patents and patent application describe the state of the art with the common methods and materials disclosed herein and further describe various elements (and methods for their manufacture) that can be used in the sensor designs disclosed herein. These include for example, U.S. Pat. Nos. 6,413,393; 6,368,274; 5,786,439; 5,777,060; 5,391,250; 5,390,671; 5,165,407, 4,890,620, 5,390,671, 5,390,691, 5,391,250, 5,482,473, 5,299,571, 5,568,806; United States Patent Application 20020090738; as well as PCT International Publication Numbers WO 01/58348, WO 03/034902, WO 03/035117, WO 03/035891, WO 03/023388, WO 03/022128, WO 03/022352, WO 03/023708, WO 03/036255, WO03/036310 and WO 03/074107, the contents of each of which are incorporated herein by reference. [0001]

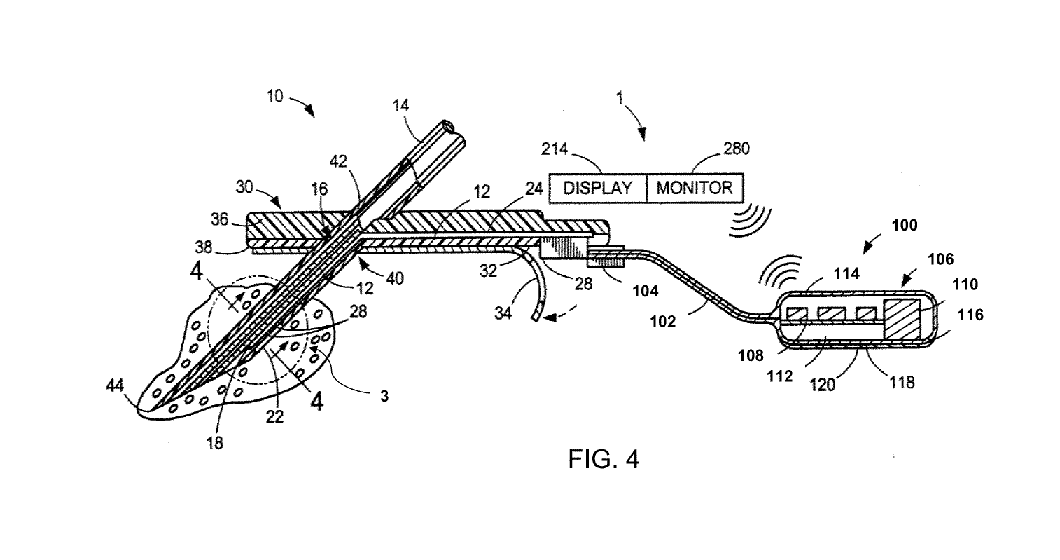

Embodiments of the invention include subcutaneous sensor insertion systems comprising sensors having the plasma deposited AP layers as disclosed herein. FIG. 4 provides a perspective view of one generalized embodiment of subcutaneous sensor insertion system and a block diagram of a sensor electronics device according to one illustrative embodiment of the invention. Additional elements typically used with such sensor system embodiments are disclosed for example in U.S. Patent Application No. 20070163894, the contents of which are incorporated by reference. FIG. 4 provides a perspective view of a telemetered characteristic monitor system 1, including a subcutaneous sensor set 10 provided for subcutaneous placement of an active portion of a flexible sensor 12, or the like, at a selected site in the body of a user. The subcutaneous or percutaneous portion of the sensor set 10 includes a hollow, slotted insertion needle 14 having a sharpened tip 44, and a cannula 16. Inside the cannula 16 is a sensing portion 18 of the sensor 12 to expose one or more sensor electrodes 20 to the user's bodily fluids through a window 22 formed in the cannula 16. The sensing portion 18 is joined to a connection portion 24 that terminates in conductive contact pads, or the like, which are also exposed through one of the insulative layers. The connection portion 24 and the contact pads are generally adapted for a direct wired electrical connection to a suitable monitor 200 coupled to a display 214 for monitoring a user's condition in response to signals derived from the sensor electrodes 20. The connection portion 24 may be conveniently connected electrically to the monitor 200 or a characteristic monitor transmitter 100 by a connector block 28 (or the like) as shown and described in U.S. Pat. No. 5,482,473, entitled FLEX CIRCUIT CONNECTOR, which is incorporated by reference.

As shown in FIG. 4, in accordance with embodiments of the present invention, subcutaneous sensor set 10 may be configured or formed to work with either a wired or a wireless characteristic monitor system. The proximal part of the sensor 12 is mounted in a mounting base 30 adapted for placement onto the skin of a user. The mounting base 30 can be a pad having an underside surface coated with a suitable pressure sensitive adhesive layer 32, with a peel-off paper strip 34 normally provided to cover and protect the adhesive layer 32, until the sensor set 10 is ready for use. The mounting base 30 includes upper and lower layers 36 and 38, with the connection portion 24 of the flexible sensor 12 being sandwiched between the layers 36 and 38. The connection portion 24 has a forward section joined to the active sensing portion 18 of the sensor 12, which is folded angularly to extend downwardly through a bore 40 formed in the lower base layer 38. Optionally, the adhesive layer 32 (or another portion of the apparatus in contact with in vivo tissue) includes an anti-inflammatory agent to reduce an inflammatory response and/or anti-bacterial agent to reduce the chance of infection. The insertion needle 14 is adapted for slide-fit reception through a needle port 42 formed in the upper base layer 36 and through the lower bore 40 in the lower base layer 38. After insertion, the insertion needle 14 is withdrawn to leave the cannula 16 with the sensing portion 18 and the sensor electrodes 20 in place at the selected insertion site. In this embodiment, the telemetered characteristic monitor transmitter 100 is coupled to a sensor set 10 by a cable 102 through a connector 104 that is electrically coupled to the connector block 28 of the connector portion 24 of the sensor set 10.

In the embodiment shown in FIG. 4, the telemetered characteristic monitor 100 includes a housing 106 that supports a printed circuit board 108, batteries 110, antenna 112, and the cable 102 with the connector 104. In some embodiments, the housing 106 is formed from an upper case 114 and a lower case 116 that are sealed with an ultrasonic weld to form a waterproof (or resistant) seal to permit cleaning by immersion (or swabbing) with water, cleaners, alcohol or the like. In some embodiments, the upper and lower case 114 and 116 are formed from a medical grade plastic. However, in alternative embodiments, the upper case 114 and lower case 116 may be connected together by other methods, such as snap fits, sealing rings, RTV (silicone sealant) and bonded together, or the like, or formed from other materials, such as metal, composites, ceramics, or the like. In other embodiments, the separate case can be eliminated and the assembly is simply potted in epoxy or other moldable materials that is compatible with the electronics and reasonably moisture resistant. As shown, the lower case 116 may have an underside surface coated with a suitable pressure sensitive adhesive layer 118, with a peel-off paper strip 120 normally provided to cover and protect the adhesive layer 118, until the sensor set telemetered characteristic monitor transmitter 100 is ready for use.

In the illustrative embodiment shown in FIG. 4, the subcutaneous sensor set 10 facilitates accurate placement of a flexible thin film electrochemical sensor 12 of the type used for monitoring specific blood parameters representative of a user's condition. The sensor 12 monitors glucose levels in the body, and may be used in conjunction with automated or semi-automated medication infusion pumps of the external or implantable type as described in U.S. Pat. No. 4,562,751; 4,678,408; 4,685,903 or 4,573,994, to control delivery of insulin to a diabetic patient.

In the illustrative embodiment shown in FIG. 4, the sensor electrodes 10 may be used in a variety of sensing applications and may be configured in a variety of ways. For example, the sensor electrodes 10 may be used in physiological parameter sensing applications in which some type of biomolecule is used as a catalytic agent. For example, the sensor electrodes 10 may be used in a glucose and oxygen sensor having a glucose oxidase enzyme catalyzing a reaction with the sensor electrodes 20. The sensor electrodes 10, along with a biomolecule or some other catalytic agent, may be placed in a human body in a vascular or non-vascular environment. For example, the sensor electrodes 20 and biomolecule may be placed in a vein and be subjected to a blood stream, or may be placed in a subcutaneous or peritoneal region of the human body.

In the embodiment of the invention shown in FIG. 4, the monitor of sensor signals 200 may also be referred to as a sensor electronics device 200. The monitor 200 may include a power source, a sensor interface, processing electronics (i.e. a processor), and data formatting electronics. The monitor 200 may be coupled to the sensor set 10 by a cable 102 through a connector that is electrically coupled to the connector block 28 of the connection portion 24. In an alternative embodiment, the cable may be omitted. In this embodiment of the invention, the monitor 200 may include an appropriate connector for direct connection to the connection portion 104 of the sensor set 10. The sensor set 10 may be modified to have the connector portion 104 positioned at a different location, e.g., on top of the sensor set to facilitate placement of the monitor 200 over the sensor set.

Another embodiment of the invention disclosed herein is a method of making a sensor apparatus for implantation within a mammal from a plurality of layered elements including adhesion promoting layers formed using plasma deposition processes. This method can comprise the steps of: providing a base layer; forming a conductive layer on the base layer, wherein the conductive layer includes an electrode (and typically a working electrode, a reference electrode and a counter electrode); forming an analyte sensing layer on the conductive layer, wherein the analyte sensing layer includes a composition that can alter the electrical current at the electrode in the conductive layer in the presence of an analyte (as does glucose oxidase in the presence of glucose); optionally forming a protein layer on the analyte sensing layer; and then forming the adhesion promoting layer on the analyte sensing layer or the optional protein layer. Subsequent layers are then deposited on this adhesion promoting layer.

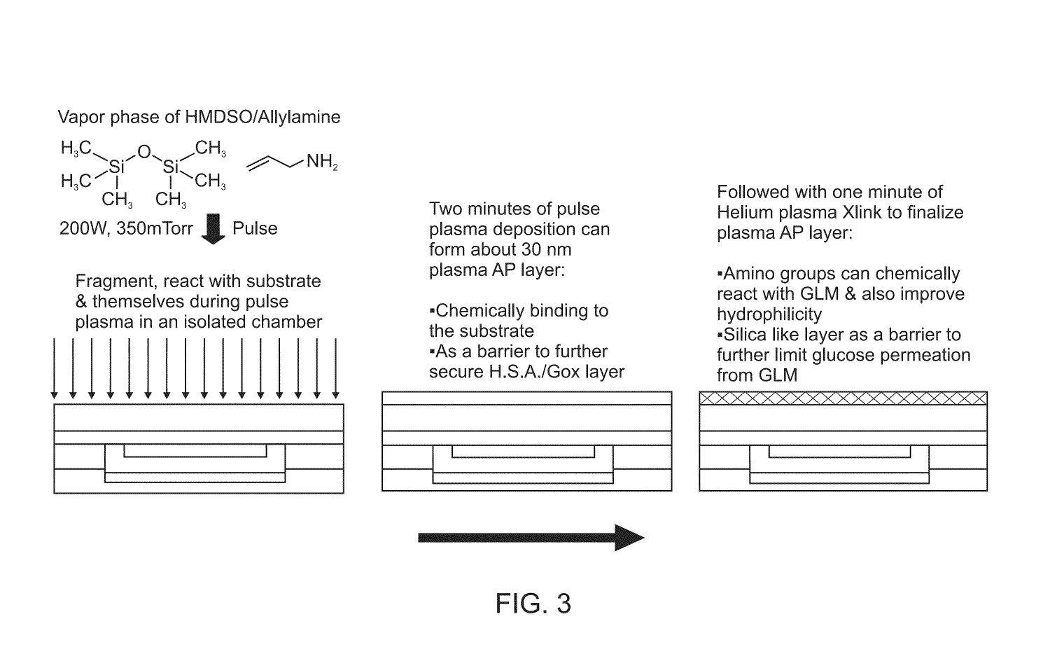

In typical embodiments of the methods of invention, an adhesion promoter layer is disposed between other functional layers in order to facilitate their contact and increase the stability of the sensor apparatus. The compositions that make up the adhesion promoter layer are selected to provide a number of desirable characteristics such as contributing to sensor stability in combination with an ability to be deposited over one or more layers of the sensor using gas plasma process. Typically such Plasma AP processes can use a commercially available systems such a PVA-TePla™ M4L chamber. One general plasma AP process includes the steps illustrated in the schematic shown in FIG. 3. One illustrative step-wise procedure for use with amperometric glucose sensors is as follows:

1. Prepare the desired underlying layers on which the AP layer is to be deposited (e.g. a protein layer comprising HSA or an analyte sensing layer comprising GOx). Pre-treat this sensor stack (e.g. up to HSA or GOx layer) with short time gas plasma (e.g. Helium plasma, O2 plasma, or continuous wave monomer plasma) to activate the surface of the substrate.

2. Create very thin film on top of the specific layer on which the AP layer is to be deposited (e.g. e.g. a protein layer comprising HSA or an analyte sensing layer comprising GOx) using HMDSO (and optionally) allylamine pulse plasma deposition (e.g. using 60 sccm of allylamine, 60 sccm of HMDSO, at 200 W, 350 mT, 2 min 10 seconds, and 30% of pulse duty cycle while pulse frequency is 20). According to different requirements, the allylamine to HMDSO ratios can be adjusted. In certain embodiments, the layer comprises 100% HMDSO and is formed using HMDSO alone in a plasma process.

Allylamine and HMDSO precursors can provide siloxane groups and amino functional groups that some conventional adhesion promoter (3-aminopropyltriethoxysilane) can also provide, but don't have problematical issues associated with some conventional AP processes, such as low vapor pressure and high sensitivity to moisture in the air during sensor fabrication. Instead of liquid phases of those two monomers, their vapor phases are used, and each vapor produces a unique plasma composition resulting in layers comprising unique surface properties. During the pulse plasma deposition process, the allylamine and HMDSO monomer vapors undergo fragmentation and reacts with the substrate and also with themselves to combine into pinhole-free films. In addition, HMDSO pulse plasma deposition generates silica like thin film layer to bind to the activated substrate, which can be a good barrier to secure a analyte modulating layer (e.g. on comprising) GOx layer underneath and to further limit analyte (e.g. glucose) permeation from a an analyte modulating layer (e.g. a GLM layer) on the top. Allylamine precursor can form relatively hydrophilic polymer membrane and also provide amino function groups to chemically bind to groups in a proximal layer (e.g. groups found in a GLM).

3. Following this plasma pulse deposition process, an appropriate plasma process (e.g. helium plasma at 200 W, 350 mTorr, for 75 seconds) can be used to crosslink the newly deposited AP layer. This process can increase the stability of the deposition. Low power short time O2 plasma (e.g. 10 W and 10 seconds) is another option of such post deposition treatments, especially for AP compositions comprising only HMDSO.

4. Following such crosslinking processes, one can then wash the plasma processed plates, for example, for 5 minutes with DI water in a wash station and then dry the plates (e.g. with spin dry equipment). Such wash steps can be used to remove undesired residual chemicals (e.g. those that are not covalently bonded).

5. After such washing/drying steps, a subsequent layer (e.g. an analyte modulating layer comprising a GLM) can be directly coated on to the treated sensor stack. In this way, an AP layer is formed without using the problematic compounds such as glutaraldehyde.

As noted above, embodiments of the invention include the steps of forming an analyte modulating layer over the plasma deposited adhesion promoting layer. Typically, the adhesion promoting layer is in direct contact with the analyte modulating layer. In such embodiments, the analyte modulating layer includes a polymeric composition that modulates the diffusion of the analyte therethrough (e.g. a glucose limiting membrane). The method can also include and forming a cover layer disposed on at least a portion of the analyte modulating layer, wherein the cover layer further includes an aperture over at least a portion of the analyte modulating layer. In certain embodiments of the invention, the analyte modulating layer comprises a linear polyurethane/polyurea polymer stabilized with a branched acrylate copolymer having a central chain and a plurality of side chains coupled to the central chain.

As discussed in detail below, the various layers of the sensor can be manufactured to exhibit a variety of different characteristics which can be manipulated according to the specific design of the sensor. Typically, a method of making the sensor includes the step of forming a protein layer on the analyte sensing layer, wherein a protein within the protein layer is an albumin selected from the group consisting of bovine serum albumin and human serum albumin. Typically, a method of making the sensor includes the step of forming an analyte sensing layer that comprises an enzyme composition selected from the group consisting of glucose oxidase, glucose dehydrogenase, lactate oxidase, hexokinase and lactate dehydrogenase. In such methods, the analyte sensing layer typically comprises a carrier protein composition in a substantially fixed ratio with the enzyme, and the enzyme and the carrier protein are distributed in a substantially uniform manner throughout the analyte sensing layer.

The disclosure provided herein includes sensors and sensor designs that can be generated using combinations of various well known techniques. The disclosure further provides methods for applying very thin enzyme coatings to these types of sensors as well as sensors produced by such processes. In this context, some embodiments of the invention include methods for making such sensors on a substrate according to art accepted processes. In certain embodiments, the substrate comprises a rigid and flat structure suitable for use in photolithographic mask and etch processes.

In this regard, the substrate typically defines an upper surface having a high degree of uniform flatness. A polished glass plate may be used to define the smooth upper surface. Alternative substrate materials include, for example, stainless steel, aluminum, and plastic materials such as delrin, etc. In other embodiments, the substrate is non-rigid and can be another layer of film or insulation that is used as a substrate, for example plastics such as polyimides and the like.

An initial step in the methods of the invention typically includes the formation of a base layer of the sensor. The base layer can be disposed on the substrate by any desired means, for example by controlled spin coating. In addition, an adhesive may be used if there is not sufficient adhesion between the substrate layer and the base layer. A base layer of insulative material is formed on the substrate, typically by applying the base layer material onto the substrate in liquid form and thereafter spinning the substrate to yield the base layer of thin, substantially uniform thickness. These steps are repeated to build up the base layer of sufficient thickness, followed by a sequence of photolithographic and/or chemical mask and etch steps to form the conductors discussed below. In an illustrative form, the base layer comprises a thin film sheet of insulative material, such as ceramic or polyimide substrate. The base layer can comprise an alumina substrate, a polyimide substrate, a glass sheet, controlled pore glass, or a planarized plastic liquid crystal polymer. The base layer may be derived from any material containing one or more of a variety of elements including, but not limited to, carbon, nitrogen, oxygen, silicon, sapphire, diamond, aluminum, copper, gallium, arsenic, lanthanum, neodymium, strontium, titanium, yttrium, or combinations thereof. Additionally, the substrate may be coated onto a solid support by a variety of methods well-known in the art including chemical vapor deposition, physical vapor deposition, or spin-coating with materials such as spin glasses, chalcogenides, graphite, silicon dioxide, organic synthetic polymers, and the like.

The methods of the invention further include the generation of a conductive layer having one or more sensing elements. Typically these sensing elements are electrodes that are formed by one of the variety of methods known in the art such as photoresist, etching and rinsing to define the geometry of the active electrodes. The electrodes can then be made electrochemically active, for example by electrodeposition of Pt black for the working and counter electrode, and silver followed by silver chloride on the reference electrode. A sensor layer such as a sensor chemistry enzyme layer can then be disposed on the sensing layer by electrochemical deposition or a method other than electrochemical deposition such a spin coating, followed by vapor crosslinking, for example with a dialdehyde (glutaraldehyde) or a carbodi-imide.

Electrodes of the invention can be formed from a wide variety of materials known in the art. For example, the electrode may be made of a noble late transition metals. Metals such as gold, platinum, silver, rhodium, iridium, ruthenium, palladium, or osmium can be suitable in various embodiments of the invention. Other compositions such as carbon or mercury can also be useful in certain sensor embodiments. Of these metals, silver, gold, or platinum is typically used as a reference electrode metal. A silver electrode which is subsequently chloridized is typically used as the reference electrode. These metals can be deposited by any means known in the art, including an electroless method which may involve the deposition of a metal onto a previously metallized region when the substrate is dipped into a solution containing a metal salt and a reducing agent. The electroless method proceeds as the reducing agent donates electrons to the conductive (metallized) surface with the concomitant reduction of the metal salt at the conductive surface.

In an exemplary embodiment of the invention, the base layer is initially coated with a thin film conductive layer by electrode deposition, surface sputtering, or other suitable process step. In one embodiment this conductive layer may be provided as a plurality of thin film conductive layers, such as an initial chrome-based layer suitable for chemical adhesion to a polyimide base layer followed by subsequent formation of thin film gold-based and chrome-based layers in sequence. In alternative embodiments, other electrode layer conformations or materials can be used. The conductive layer is then covered, in accordance with conventional photolithographic techniques, with a selected photoresist coating, and a contact mask can be applied over the photoresist coating for suitable photoimaging. The contact mask typically includes one or more conductor trace patterns for appropriate exposure of the photoresist coating, followed by an etch step resulting in a plurality of conductive sensor traces remaining on the base layer. In an illustrative sensor construction designed for use as a subcutaneous glucose sensor, each sensor trace can include three parallel sensor elements corresponding with three separate electrodes such as a working electrode, a counter electrode and a reference electrode.

Portions of the conductive sensor layers are typically covered by an insulative cover layer, typically of a material such as a silicon polymer and/or a polyimide. The insulative cover layer can be applied in any desired manner. In an exemplary procedure, the insulative cover layer is applied in a liquid layer over the sensor traces, after which the substrate is spun to distribute the liquid material as a thin film overlying the sensor traces and extending beyond the marginal edges of the sensor traces in sealed contact with the base layer. This liquid material can then be subjected to one or more suitable radiation and/or chemical and/or heat curing steps as are known in the art. In alternative embodiments, the liquid material can be applied using spray techniques or any other desired means of application. Various insulative layer materials may be used such as photoimagable epoxyacrylate, with an illustrative material comprising a photoimagable polyimide available from OCG, Inc. of West Paterson, N.J., under the product number 7020.

Subsequent to treatment of the sensor elements, one or more additional functional coatings or cover layers can then be applied by any one of a wide variety of methods known in the art, such as spraying, dipping, etc. Some embodiments of the present invention include an analyte modulating layer deposited over the enzyme-containing layer. In addition to its use in modulating the amount of analyte(s) that contacts the active sensor surface, by utilizing an analyte limiting membrane layer, the problem of sensor fouling by extraneous materials is also obviated. As is known in the art, the thickness of the analyte modulating membrane layer can influence the amount of analyte that reaches the active enzyme. Consequently, its application is typically carried out under defined processing conditions, and its dimensional thickness is closely controlled. Microfabrication of the underlying layers can be a factor which affects dimensional control over the analyte modulating membrane layer as well as exact the composition of the analyte limiting membrane layer material itself. In this regard, it has been discovered that several types of copolymers, for example, a copolymer of a siloxane and a nonsiloxane moiety, are particularly useful. These materials can be microdispensed or spin-coated to a controlled thickness.

In some embodiments of the invention, the sensor is made by methods which apply an analyte modulating layer that comprises a hydrophilic membrane coating which can regulate the amount of analyte that can contact the enzyme of the sensor layer. For example, the cover layer that is added to the glucose sensors of the invention can comprise a glucose limiting membrane, which regulates the amount of glucose that contacts glucose oxidase enzyme layer on an electrode. Such glucose limiting membranes can be made from a wide variety of materials known to be suitable for such purposes, e.g., silicones such as polydimethyl siloxane and the like, polyurethanes, cellulose acetates, Nafion, polyester sulfonic acids (e.g. Kodak AQ), hydrogels or any other membrane known to those skilled in the art that is suitable for such purposes. In certain embodiments of the invention, the analyte modulating layer comprises a linear polyurethane/polyurea polymer stabilized with a branched acrylate copolymer having a central chain and a plurality of side chains coupled to the central chain, wherein at least one side chain comprises a silicone moiety.

Embodiments of the invention include one or more exterior cover constituents which are typically electrically insulating protective constituents (see, e.g. element 106 in FIG. 2). Typically, such cover constituents are disposed over at least a portion of the analyte modulating constituent.

Acceptable polymer coatings for use as the insulating protective cover constituent can include, but are not limited to, non-toxic biocompatible polymers such as silicone compounds, polyimides, biocompatible solder masks, epoxy acrylate copolymers, or the like.

### EXAMPLES

### Example 1

### Illustrative Working Embodiments of the Invention

A plasma is a gas energized to a state of electrical conductivity in which a significant percentage of the atoms or molecules are ionized. In this state electrons, ions, radicals, excited neutrals, photons, and electric and magnetic fields are present. The collective properties of these components constitute the plasma phenomenon. Plasma-enhanced chemical vapor deposition is a process that uses plasma technology to deposit thin films from a gas state (vapor) to a solid state on a substrate. Working embodiments of the invention utilize plasma deposition technologies to form adhesion promoting layers useful in layered sensor structures, including a dual precursor plasma AP material deposition process useful in implantable glucose sensor fabrication. Such processes avoid certain problems associated with wet chemistry AP processes and provides an environment friendly alternative to conventional methods.

Using, for example, a TePla M4L Plasma Processing System, advanced dry plasma AP materials and processes have been developed and characterized as useful in sensor fabrication processes as well as having a biocompatibility profile that is compatible with in vivo implantation. Plasma AP has many advantages over current wet chemistry AP process, such as automatic process potential, significantly reducing process time, eliminating toxic glutaraldehyde and reducing daily chemical wastes.

### Illustrative In Vitro and In Vivo Tests:

The following tests were conducted on amperometric glucose sensors having the following layered elements disposed over each other in the following order: a base layer, a conductive layer comprising electrodes, a analyte sensing layer comprising GOx, a protein layer comprising HSA, a plasma deposited adhesion promoting layer comprising HMDSO and an analyte modulating layer comprising a glucose limiting membrane (GLM).

A sensor structures (up to HSA layer) were formed by methods that included a plasma AP process as disclosed herein. After rinsing and drying the plates following the addition of the AP layer, a GLM was applied over the AP layer via slot coating and then baked according to conventional protocols. As noted below, after sterilization, those plasma AP treated sensors were evaluated using BTS and SITS in vitro tests as well as in vivo tests.

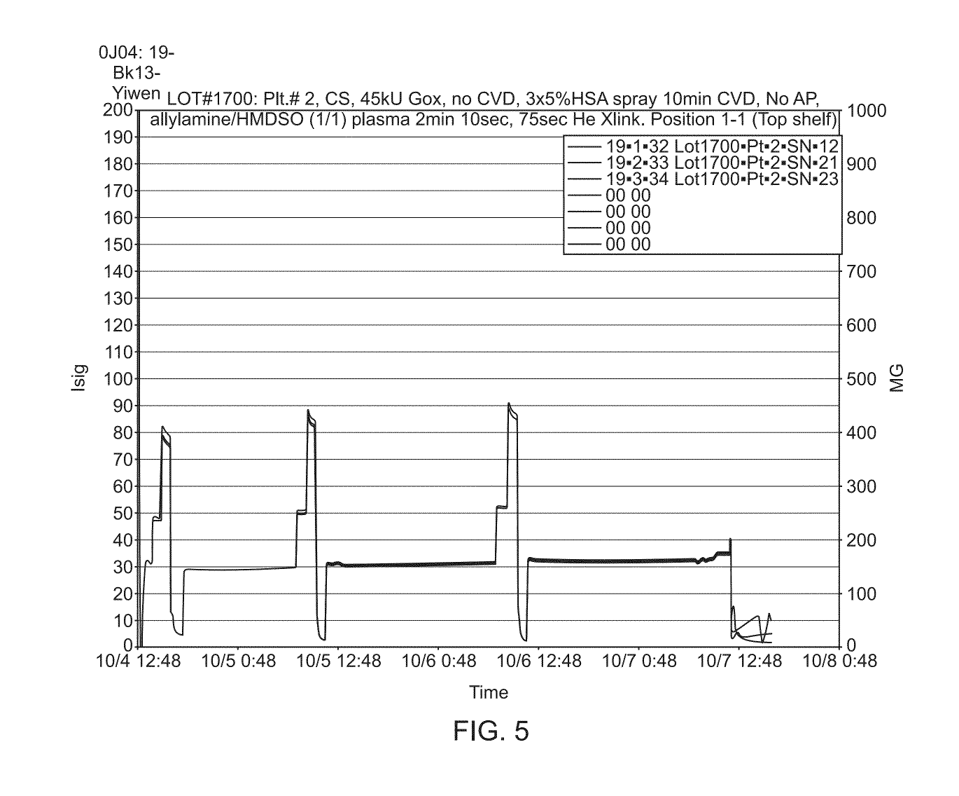

FIGS. 5-8 provide data from tests of these glucose sensors in in vitro testing systems that are designed to replicate in vivo conditions. These systems include a bicarbonate buffer testing system “BTS” and other in vitro sensor test systems (SITS) designed to mimic in vivo glucose oxidase sensor conditions, including stoichiometrically high levels of oxygen relative to glucose. In these systems, sensor current is measured periodically in the presence of known concentrations of glucose. As is known in the art, in glucose oxidase based sensors, glucose values can be correlated with Isig, that is sensor current (in μA). For example, when used in in vivo environments such as an interstitial space, these sensors can be used to measure glucose using calculations based on a formula IG=Isig×CAL, where IG is interstitial glucose value (in mmol/l or mg/dl), Isig is sensor current (in μA) and CAL is calibration factor (in mmol/l/μA or mg/dl/μA). The data presented in these graphs shows that sensor Isig of the various working sensor embodiments appropriately correlates with different glucose concentrations in in vitro tests in multiple systems designed to mimic in vivo conditions.

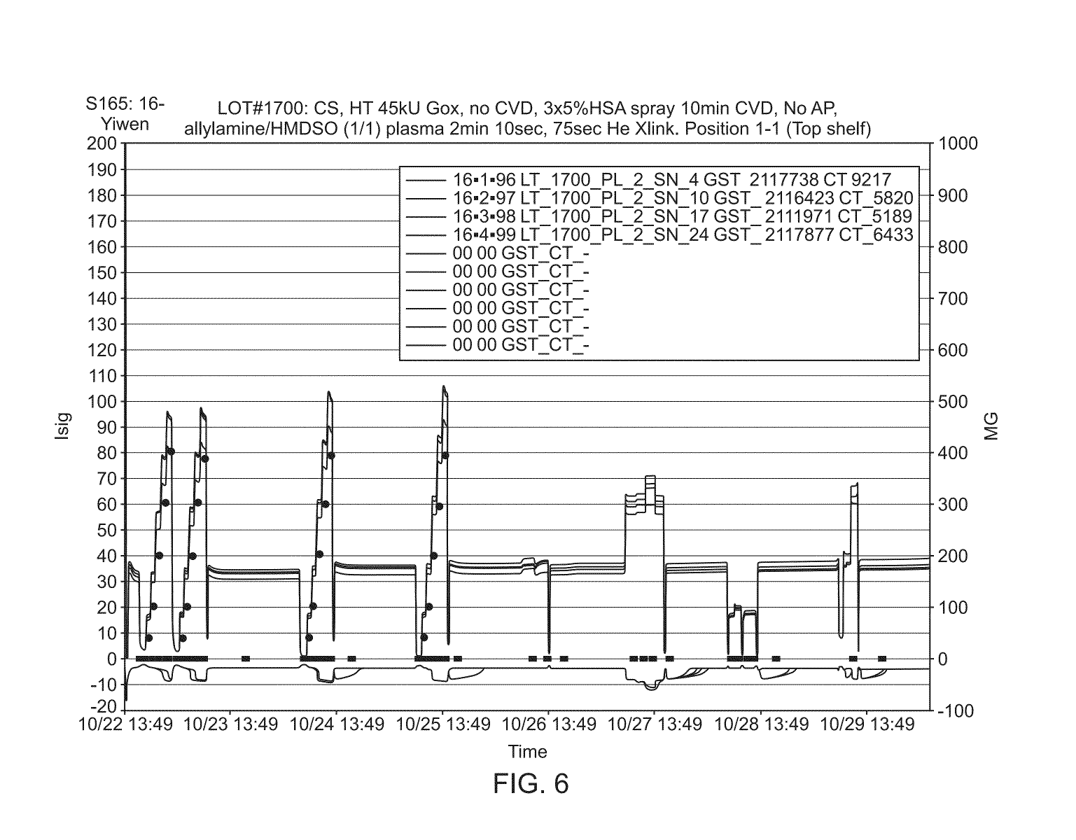

FIG. 5 provides a graph of BTS data (Isig over periods of time) using sensors constructed to include a plasma deposited AP layer comprising: HMDSO/allylamine (1:1 ratio, plasma process comprising equal vapor flow rate of two precursors). This 3-day-long in vitro testing result indicated that those sensors had good starting Isig at 100 mg/dl glucose level, very small sensor to sensor variations and stable Isig (no drift up issues) even at the end of the test, suggesting the plasma AP was comparable to if not better than the wet chemistry AP. Seven days of SITS test further confirmed the preliminary BTS results (FIG. 6). FIG. 6 provides a graph of SITS data (Isig over periods of time) using sensors constructed to include a plasma deposited AP layer comprising: HMDSO/allylamine. This 7-day-long standard sensor in vitro test result indicated those sensors passed 4 calibration tests at different glucose levels, an oxygen response test, a temperature response test, and Isig stability test with limited sensor-to-sensor variation.

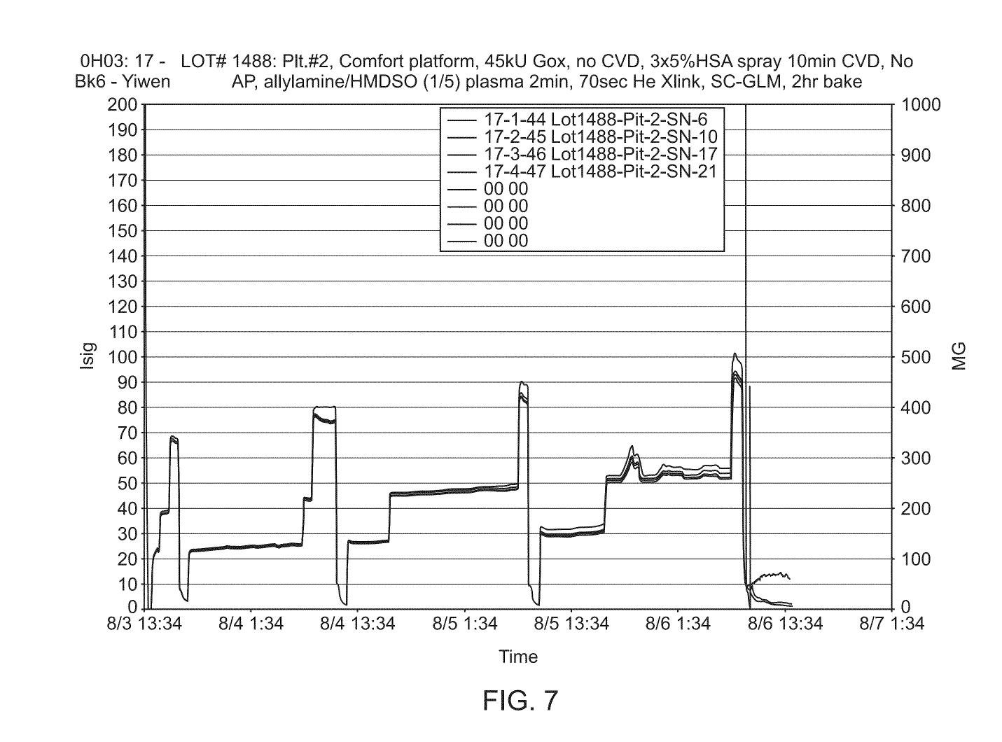

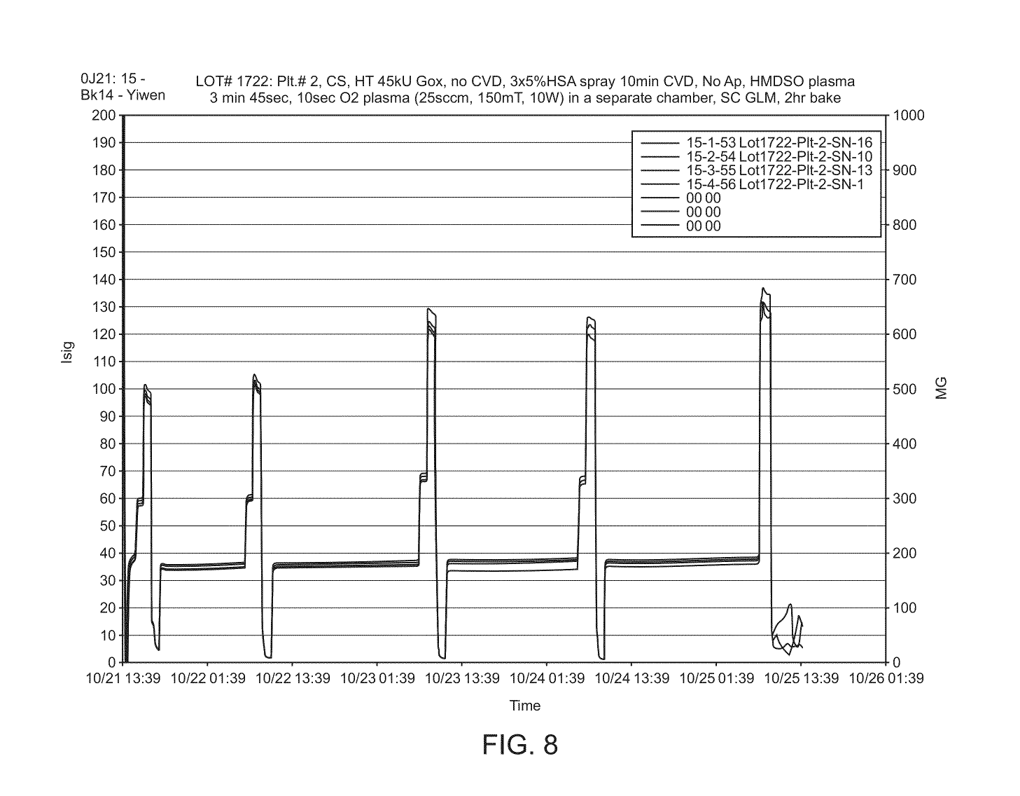

HMDSO/allylamine (at a ratio of 1:1) plasma AP was tested and confirmed to provide very good and highly reliable results. However, the plasma AP process can be varied according to the desired result (e.g. a desired ratio of HMDSO/allylamine). FIG. 7 shows that a BTS result of HMDSO/allylamine at a ratio of 5:1. An alternative to combinations of HMDSO/allylamine plasma AP processes is HMDSO (one precursor) plasma AP process. This HMDSO solo precursor plasma AP deposition process can include a 2nd step, that is, the use O2 plasma to activate the HMDSO plasma deposited material prior to applying a GLM layer (See FIG. 8). Typical parameters for this process include: HMDSO (80 sccm) plasma pulse at 200 Watt and 350 mTorr for about 4 minutes (3 minutes 45 seconds to 4 minutes 15 seconds, duty cycle=30, Frequency=1), followed with O2 plasma at about 10 Watts for about 10 seconds.

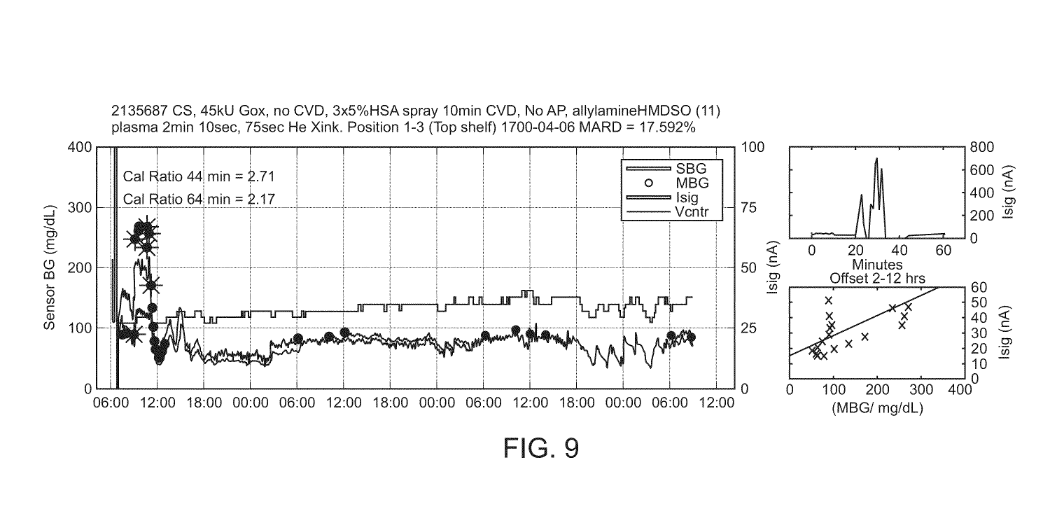

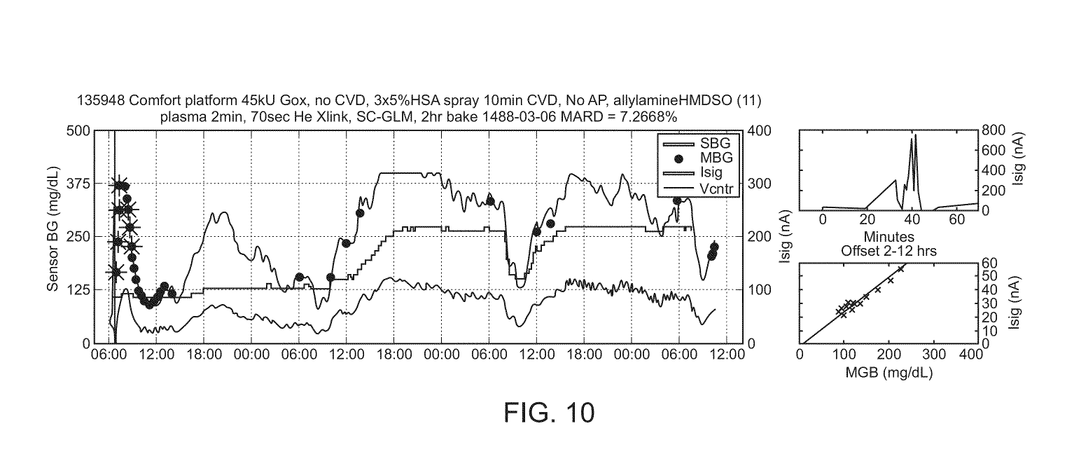

FIGS. 9, 10, and 12 provide data from tests of these glucose sensors in vivo. As shown by the data presented in these figures plasma AP sensors have been tested in both non-diabetic and diabetic dogs with excellent results. FIG. 9 shows data from plasma AP sensors in a non diabetic dog, data that shows the Isig of the sensor matches well with the corresponding blood gas measurement results. Additionally, after 3 days of implantation and test, the sensor Isig is still strong. FIG. 10 shows that the sensor follows the actual glucose level change very well in a diabetic dog. Other in vitro and in vivo testing procedures used to evaluate the functionality and biocompatibility of implantable glucose sensors are discussed, for example, in Koschwanez et al., Biomaterials. 2007 28(25): 3687-3703.

The data from these tests in vitro and in vivo tests shows that sensors formed with these plasma deposited AP layers disclosed herein exhibit functional characteristics that are comparable, if not better than, sensors formed with conventional wet chemistry AP layers. The data illustrates the surprising observation that hexamethyldisiloxane alone as well as hexamethyldisiloxane and allylamine combined in a ratio of between 5:1 and 1:1 not only promotes adhesion between layers having very different material properties (e.g. adhesion between a layer comprising a glucose limiting membrane comprising a linear polyurethane/polyurea polymer and a protein layer consisting of an albumin or a protein layer comprising an albumin combined with glucose oxidase) and further allows layered glucose sensors to function at least as well as conventional layered glucose sensors. Without being bound by a specific scientific theory or mechanism of action, it is believed that van der Waals forces (or van der Waals interactions) may contribute to the adhesive functionality observed with these materials.

### Plasma AP Uniformity and Scale-Up Study

A series of plasma AP uniformity and scale-up studies have been conducted in order to illustrate that this process can be adapted for mass production. During those studies, up to 3 lots of sensor plates were successfully processed with a single plasma AP run. The variations within one run and between different runs were also checked with several different characterization methods.

### BTS & SITS Studies

Tests of in a plasma chamber shows that the thickness of the plasma AP coating can be modulated in a number of ways including sensor placement within the chamber. Observations using visual inspection under microscope that show that the plasma AP coating on sensors placed on a bottom shelf is a little bit lighter and thinner than the coating on sensors placed on a top or a middle shelf. modulated AP sensors within a single run. Sensors treated at different location of the plasma chamber within a single run, i.e. top shelf, middle shelf and bottom shelf show no significant differences. Observations of the bottom shelf group, show that the corresponding starting Isig at 100 mg/dl glucose is a little bit higher than other groups.

Considering each shelf in the M4L plasma chamber can held up to twelve 2.5 by 2.5 inch Sensor plates, one single plasma AP run still can process multiple sensor lots even without using the bottom shelf.

### Ellipsometry and Plasma AP Thickness Studies

As part of the plasma AP uniformity study, 3 inch silicon wafers plates were put in the M4L plasma chamber to receive a plasma AP treatment. This AP process was repeated 3 times by three different operators. The thicknesses of the plasma coatings were measured with an ellipsometer (M2000F, J. A. Woollam). Three regions of each wafer were measured for thickness. Table 1 below shows that the thickness variation between different runs is very limited, providing evidence of a very high consistency/repeatability of the plasma AP process. In addition, the intra wafer standard deviation (SD) is small; providing evidence that the plasma AP coating on each wafer is pretty uniform.

TABLE 1

| Different | | | | | | |
|---|---|---|---|---|---|---|
| plasma AP | | | | | | |
| runs (same | | | | | | |
| sample | | | | | | |
| location- | | | | | | |
| regular | | Thickness-1 | Thickness-2 | Thickness-3 | Ave | |
| position) | Plasma AP process | (nm) | (nm) | (nm) | (nm) | SD |
| 1^{st} run | 200 W, | 31 | 30.9 | 31.1 | 31.0 | 0.1 |
| 2^{nd} run | allylamine/HMDSO | 31.9 | 31.9 | 32.1 | 32.0 | 0.1 |
| 3^{rd} run | (1/1) Plasma pulse | 31.6 | 31.9 | 31.7 | 31.7 | 0.2 |
| | deposition for 2 min | | | | | |
| | 10 sec, Helium | | | | | |
| | plasma Xlink @200 W | | | | | |
| | for 75 sec, Rinse/dry | | | | | |

### Fourier Transform Infrared Spectroscopy (FTIR) Study

Twelve KBr discs==plates were put into the M4L plasma chamber (M24748) with 4 discs per shelf. A HMDSO/allylamine plasma AP process with extended deposition time (11 minutes in place of typical 2 minutes) was conducted to ensure a thickness of the coating suitable for a FTIR scan (Nexus 670 FT-IR, M10681).

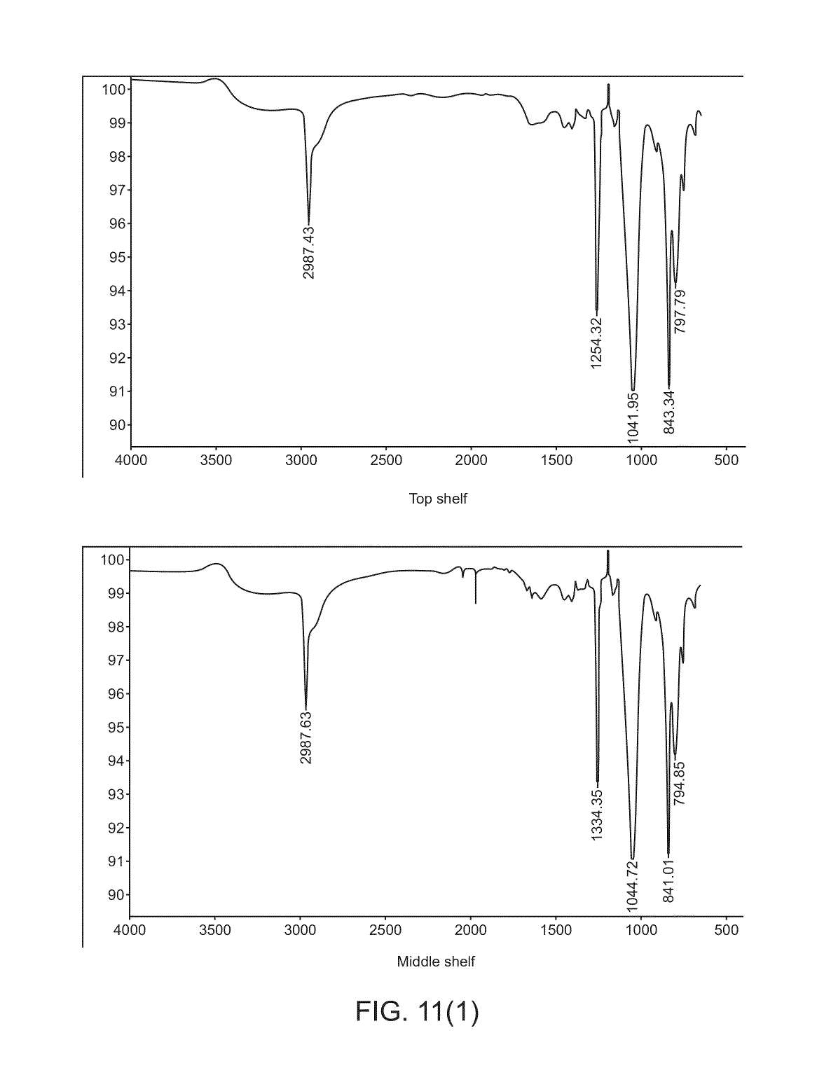

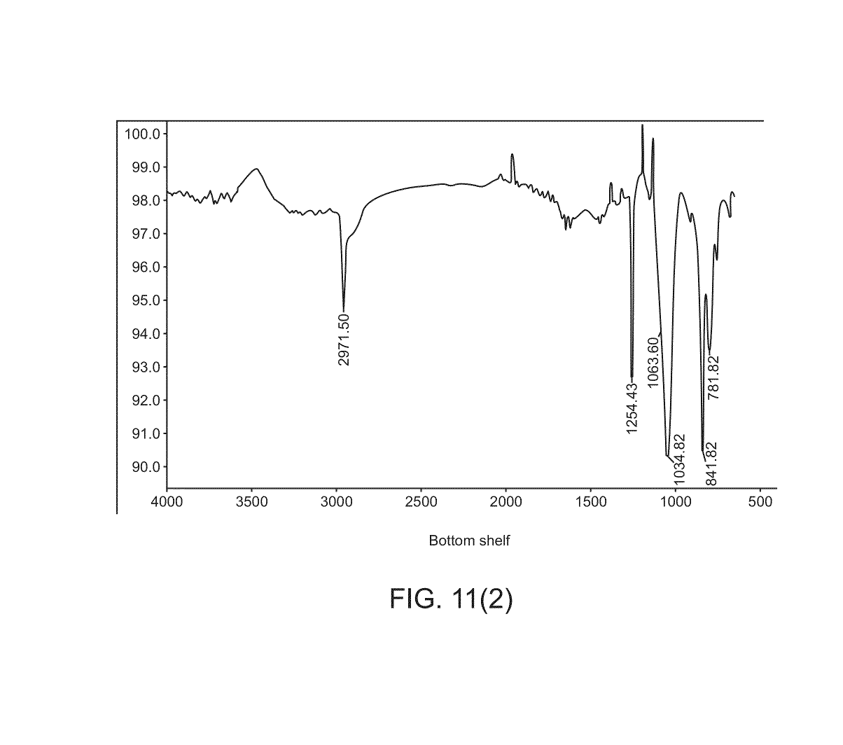

FIG. 11 includes 3 FTIR spectra respectively obtained from samples related to three different shelves in a single run. There is no obvious different between those graphs, data which provides evidence that the uniformity of the plasma AP process was good. The bands at 2958 and 2901 cm-1 are due to methyl and methylene groups. The band at 1254 cm-1 is due to SiCH3, and the three bands at 841, 797, and 754 cm-1 are due to Si(CHx)x. These signals are intense and indicate that the methylene and methyl groups on the silicons are intact. The band at 1045 cm-1 is another Si—O—Si band, and the lack of splitting provides evidence that the sample is cross linked.

### X-Ray Photoelectron Spectroscopy (XPS) Surface Analysis Study

The variability of the plasma AP deposition process as a function of position in the chamber has also been studied. The surface chemistry as measured by X-ray photoelectron spectroscopy (XPS, Physical Electronics VersaProbe 5000) will be used to quantify the variability because XPS is one of the most sensitive surface analysis tools. The depth of analysis of this technique is on the order of 75 Å. The measurements were seen to be stable for at least 25 min under the X-ray beam and neutralization. The samples in this study included 9 Plates from Lot 1733 (Top Shelf), 9 Plates from Lot 1769 (Middle Shelf), and 9 plates from Lot 1770 (Bottom Shelf).

Additional information can be extracted from XPS survey spectra beyond quantifying the peaks for elemental composition. The background of XPS spectra are created by inelastically scattered photoelectrons and Auger electrons, so the shape of the background is sensitive to the film structure and thickness. There was decent reproducibility in the overall shape of the spectra across plates, suggesting similar film layer structure in the top 10 nm. Lots 1733 (Top shelf) and 1769 (Middle shelf) were more similar to each other, while 1770 (Bottom shelf) was subtly different. There was less Si, and the background of the N shows some buried character, likely N from the protein substrate showing through.

### Comparing Different AP Processes in Parallel

The plasma AP is summarized and compared with regular wet Chemistry AP in parallel (Table 2 below). Plasma AP has several advantages over regular AP, such as significantly reduced process time, eliminating toxic glutaraldehyde and related CVD system, avoiding daily chemical wastes of wet chemistry AP process, etc.

### Applications to Other Sensor Platforms

Plasma AP recipes and processes can be adapted for use with a wide variety of sensor structures and/or sensor materials. In addition to the sensor platforms discussed above, a variety of plasma AP processes and recipes have been applied to other sensors, including sensors with more or less layers in various orders, sensors comprising layers made from a variety of different materials, sensors comprising multiple electrodes disposed in a distributed pattern, wire-based sensors etc. etc.

One example of a modified HMDSO AP process (“HMDSO-2A”) designed for sensors with distributed electrodes/wire-based sensors included the following parameters: HMDSO (80 sccm) plasma pulse at 200 Watt and 350 mTorr for about 4 minutes (3 minutes 45 seconds to 4 minutes 15 seconds, duty cycle=50, Frequency=20), followed with O2 plasma at about 10 Watts for about 10 seconds.

Those of skill in the art will understand that a variety of processes for forming the plasma deposited layers can be used following art accepted methodologies (see, e.g. Yoshinari et al., Biomedical Research 27(1) 29-36 (2006); Harsch et al., Journal of Neuroscience Methods 98 (2000) 135-144; and The Challenge of Plasma Processing—Its Diversity, Larner et al., Medical Device Materials II: Proceedings of the Materials & Processes for Medical Devices Conference 2004 (ASM International) 2005, Pages: 91-96, the contents of each of which are incorporated by reference.

### Biocompatibility

In vitro cytotoxicity testing confirms the biocompatibility of the sensor compositions disclosed herein. For example, in vitro cytotoxicity testing was performed on various formulations of Plasma AP including: HMDSO, HMDSO/Allylamine (5 min Rinse) and HMDSO/Allylamine (No Rinse). In these tests, sensors were fabricated using layers having known biocompatible sensor chemistries and where a conventional adhesion promoter layer was substituted with a plasma AP layers as disclosed herein. After plates were fabricated, the polyimide was laser cut along the edges of the plate, and plates were packaged in individual pouches and sent for regular E-Beam sterilization. The following processes all produce materials that are biocompatible (e.g. suitable for use with sensors that are implanted in vivo):

HMDSO sample: HMDSO plasma pulse for 7 minutes at 200 Watt and 350 mTorr, followed with 10 seconds of O₂ plasma at 10 Watt (reflecting 65% HMDSO deposition time increase comparing to the regular HMDSO plasma AP process).

HMDSO/Allylamine (No Rinse) sample: HMDSO/Allylamine (1/1) plasma pulse for 4 minutes at 200 Watt and 350 mTorr, followed with helium plasma cross link at 200 Watt (reflecting 85% HMDSO/Allylamine deposition time increase comparing to the regular HMDSO/Allylamine plasma AP process).

HMDSO/Allylamine (5 min Rinse) sample: HMDSO/Allylamine (1/1) plasma pulse for 4 minutes at 200 Watt and 350 mTorr, followed with helium plasma cross link at 200 Watt (reflecting 85% HMDSO/Allylamine deposition time increase comparing to the regular HMDSO/Allylamine plasma AP process).

TABLE 2
Comparison of Plasma AP, DSAP/CVD, DSAP/Static cross link in parallel

| Steps within each | | | DSAP/Static cross |
|---|---|---|---|
| process | Plasma AP | DSAP/CVD | link |
| Preparing 10% | | 15 minutes | 15 minutes |
| APTES solution | | | |
| Spin coating AP one | | 15 minutes | 15 minutes |
| by one | | | |
| Process in CVD | | At least 10 minutes | 2 hours 10 minutes |
| chamber (4 by 4 | | x3 | |
| plates) or with static | | | |
| cross link (a whole | | | |
| lot) | | | |
| Plasma AP for a | At most 10 minutes | | |
| whole lot | (including plasma | | |
| (Note: PVA-TePla | coating/cross link, | | |
| chamber can | pump downs and | | |
| comfortably held 2 | vents) | | |
| lots) | | | |
| Rinse | 5 minutes | 5 minutes | 5 minutes |
| Dry | 5 minutes | 5 minutes | 5 minutes |
| Notes about | 1. HMDSO and | 1. Fresh APTES | 1. Fresh APTES |
| chemicals used in | allylamine stored in | solution is needed | solution is needed |
| each AP process | stainless steel in | and only good within | and only good within |
| | vacuum state - safe | 4 hours. | 4 hours. |
| | and no moisture | 2. The dwell time | 2. The dwell time |
| | concern. | between spin-coating | between spin-coating |
| | 2. Very limited | and the following | and the following |
| | amount is consumed | cross link step | cross link step |
| | for each plasma AP | cannot exceed 8 | cannot exceed 8 |
| | process, so a cylinder | minutes. | minutes. |
| | of 100 ml of each | 3. Plate is coated one | 3. Plate is coated one |
| | can normally be used | by one, so the | by one, so the |
| | for about 4 month | difference between | difference between |
| | for R&D use. No | the 1^{st} run and the | the 1^{st} and the last of |
| | chemical waste is | last of a lot is | the lot is obvious. |
| | generated in this | obvious. | 4. Glutaraldehyde is |
| | process. | 4. Glutaraldehyde is | necessary for the |
| | | necessary for the | cross link, and has to |
| | | cross link, and has to | be replaced after |
| | | be replaced after 20 | several runs. The |
| | | CVD runs. The | equipment and |
| | | glutaraldehyde | process have to be in |
| | | concentration for the | a chemical hood. |
| | | 1^{st} run and 20^{th} runs | 5. Lots of APTES |
| | | are significantly | and glutaraldehyde |
| | | different. | wastes are generated |
| | | 5. Lots of APTES | every day. |
| | | and glutaraldehyde | |
| | | wastes are generated | |
| | | every day. | |
| Total time to | 20 minutes | At least 1 hour 10 | 2 hours 50 minutes |
| process 1 lot | | minutes | |
| Total time to | 20 minutes | At least 2 hours 10 | 5 hours 40 minutes |
| process 2 lots | | minutes | |

Embodiments of the invention are recited in the following claims.

1. A method of making an analyte sensor apparatus comprising:
providing a base layer;
forming a conductive layer over the base layer, wherein the conductive layer includes a working electrode;
forming an analyte sensing layer over the conductive layer, wherein the analyte sensing layer includes a composition that can alter the electrical current at the working electrode in the conductive layer in the presence of an analyte;
forming an adhesion promoting layer over the analyte sensing layer, wherein the adhesion promoting layer comprises hexamethyldisiloxane and is formed over the analyte sensing layer using a plasma vapor deposition process; and
forming an analyte modulating layer over the adhesion promoting layer.

2. The method claim 1, wherein the adhesion promoting layer comprises allylamine and is formed over the analyte sensing layer using a dual plasma vapor deposition process.

3. The method of claim 2, wherein the hexamethyldisiloxane and the allylamine are disposed in the adhesion promoting layer in a ratio of between 5:1 and 1:1.

4. The method of claim 1, further comprising performing a pretreatment step on a layer over which the adhesion promoting layer is deposited, wherein the pretreatment step comprises exposure to a pretreating gas plasma.

5. The method of claim 1, further comprising performing a crosslinking step on the adhesion promoting layer after it is deposited, wherein the crosslinking step comprises exposure to a crosslinking gas plasma.

6. The method of claim 5, wherein the crosslinking gas plasma comprises a Helium plasma or an Oxygen plasma.

7. The method of claim 5, further comprising performing a wash step on the analyte sensor following the crosslinking step and prior to forming the analyte modulating layer over the adhesion promoting layer.

8. The method of claim 1, wherein the plasma vapor deposition process is a pulse deposition process.

9. The method claim 1, further comprising forming a protein layer over the analyte sensing layer, and forming the adhesion promoting layer over the protein layer.

10. An analyte sensor apparatus comprising:
a base layer;
a conductive layer disposed over the base layer wherein the conductive layer includes a working electrode;
an analyte sensing layer disposed over the conductive layer, wherein the analyte sensing layer detectably alters the electrical current at the working electrode in the conductive layer in the presence of an analyte;
an adhesion promoting layer disposed over the analyte sensing layer, wherein the adhesion promoting layer comprises hexamethyldisiloxane; and
an analyte modulating layer disposed over the analyte sensing layer, wherein the analyte modulating layer modulates the diffusion of the analyte therethrough.

11. The analyte sensor apparatus of claim 10 wherein the adhesion promoting layer comprises allylamine.

12. The analyte sensor apparatus of claim 11, wherein the adhesion promoting layer comprises hexamethyldisiloxane and allylamine combined in a ratio from 5:1 to 1:1.

13. The analyte sensor apparatus of claim 12, wherein the hexamethyldisiloxane and allylamine are covalently crosslinked together.

14. The analyte sensor of claim 11, wherein the analyte modulating layer comprises an isocyanate compound and the isocyanate is covalently coupled to the allylamine.

14. The analyte sensor apparatus of claim 10, wherein the adhesion promoting layer has an average thickness of less than 60, 50 or 40 nanometers.

15. The analyte sensor apparatus of claim 10 further comprising a protein layer disposed over the analyte sensing layer, wherein the adhesion promoting layer is disposed over the protein layer.

16. The analyte sensor apparatus of claim 15, wherein the protein layer comprises bovine serum albumin or human serum albumin.

17. The analyte sensor apparatus of claim 10, wherein the analyte sensing layer comprises an enzyme selected from the group consisting of glucose oxidase, glucose dehydrogenase, lactate oxidase, hexokinase and lactose dehydrogenase.

18. The analyte sensor apparatus of claim 10, wherein the adhesion promoting layer is in direct contact with materials in the protein layer on a first side and in direct contact with materials in the analyte modulating layer on a second side.

19. The analyte sensor of claim 10, wherein the sensor is coupled to a structure adapted to be implanted in vivo.

20. A method of sensing an analyte within the body of a mammal, the method comprising:
implanting an analyte sensor of claim 10 in to the mammal;
sensing an alteration in current at the working electrode in the presence of the analyte; and
correlating the alteration in current with the presence of the analyte, so that the analyte is sensed.
